SEQUENCE LISTING (7 sequences)

SEQ ID NO: 1 - protein, 266 aa, Mus musculus
MGSTQSVSGTPARPLPRNKQVARVADPRSPSAGIQRTPIQVESSPQPSLPAEQLNGLKQAQDPDPRSPTLGIARTPMKISGPDPQCSLVKELSEVLETEASESISSPELALPRETPLFYDLDLSSDPQLSPEDQLLPWSQAELDPKQVFTKEEAKQSAETIAASQNSDKPSRDPETPQSSGSKRSRRKANSKVLGRSPLTILQDDNSPGTLTLRQGKRPSALSENVKDLKEGVVLGTGRFLKAGGGAREPNQDHDKENQHFALLES

SEQ ID NO: 2 - protein, 268 aa, Homo sapiens
MGSAKSVPVTPARPPPHNKHLARVADPRSPSAGILRTPIQVESSPQPGLPAGEQLEGLKHAQDSDPRSPTLGIARTPMKTSSGDPPSPLVKQLSEVFETEDSKSNLPPEPVLPPEAPLSSELDLPLGTQLSVEEQMPPWNQTEFPSKQVFSKEEARQPTETPVASQSSDKPSRDPETPRSSGSMRNRWKPNSSKVLGRSPLTILQDDNSPGTLTLRQGKRPSPLSENVSELKEGAILGTGRLLKTGGRAWEQGQDHDKENQHFPLVES

SEQ ID NO: 3 - protein, 363 aa, Xenopus laevis
MGSAESKAQVTPSRPLRNHLLSRVNDPRSPTSGIPRTPIEVGESPRNTPQTVKEEEEEIPDSPEIFDPRSPTNGITRTPLRPPIHAVLNNLAKQLSEVFVAEDSSTEGGPLGFTGPEATNLERQVVESQTAPPAGEHVNDHEVEPSVEKAETQIDLEVCPGVEKVKSPIAEMLETLNDQEESPIAETLETMNDQEESPIAETMNDQEESPIAETLENLNDQAESPIAETLENLNDQAESPIAEMLDTLNDQEPVAVAQSVVSTESTQATGQQQKTRGKSPRSSGVKNVRQRPRKALLSSSSGRSPLRILQEDNSPNTNTQHRQAKKLSFQSEPALPHRALKISHPNWESSLNKENAEYGHSNS

SEQ ID NO: 4 - DNA, 1426 bp, Mus musculus
gctctagctggtgttcgggcagaggtcgagctggggcccgccgggcccgtccccagccccagtggagatgggttcgactcagagcgtctcaggcactccagcacggcctctgccacgcaacaagcaagtggctcgagtagcagaccctcgttcacctagtgctggcatccagcgcactcctattcaggtagagagctctccacagccaagcctaccagcagaacagctgaacggtctcaaacaggcacaagacccagatccccgctctcctactcttggcattgcacggacacccatgaagatcagtggtccagaccctcagtgctcactggtgaaagagctgagcgaagtattggagacagaagcgtcggaatcgatttcctccccagagcttgctctgccccgggaaacgcctttattttatgacctggacctgtcttcagatcctcagttatcccctgaggaccagttactgccttggagccaggctgaactcgatcccaaacaggtgtttaccaaggaggaagccaaacaatccgcagaaactatagctgccagccagaactcagacaagccctccagagacccagagactccccagtcctcaggttctaagcgcagcagacgaaaagcaaacagcaaggttctagggaggtcccctctcaccatcctgcaggatgacaactcccctgggaccttgacactacgacagggtaagcggccttctgccctcagtgagaacgttaaggacctaaaggaaggagtcgttcttggaactggaagatttctcaaagctggaggaggagcacgggagccaaaccaggaccacgacaaggaaaatcagcattttgccttgttggagagctagggctgtgctggagtcatccccaagggcaccaaagaaagccctgttctgattcctctcaggcaccaactctggtacttggacatcgttcttttaaaatgttttatgtgtttcctgtatattaaagtagatgattttaaatgatgctttaagaagttagagcaaagctacaccgggtcttgtgtgtatagaatggcatcctaggggcctgtgagacctggatagatctctctcctgccacttctgagcctcctttatgtagcttaagctttgatgtccccagagctctgcctttggccctcttcacttcttgtttgggtttagctattgcttacctgcttgtaatttccaggtccccctctcgacataaacactcaacgttagttcccctaaaataagattcatgatctttccctcattgcctacgcctctccctgtggctggggaaaggtggtcatctcctagctactcaagcctaagcctcggctttgggcatcatattgaacatgacgtgtctgtgaggttgtcactagggcctggtgattatatattcttgtcgtc

SEQ ID NO: 5 - DNA, 1139 bp, Homo sapiens
ccacgagctgttgtgcatccagaggtggaattggggcccggcattccctcctcgtcccgggctggcccttgcccccaccctgcaactcctggttgagatgggctcagccaagagcgtcccagtcacaccagcgcggcctccgccgcacaacaagcatctggctcgagtggcggacccccgttcacctagtgctggcatcctgcgcactcccatccaggtggagagctctccacagccaggcctaccagcaggggagcaactggagggtcttaaacatgcccaggactcagatccccgctctcctactcttggtattgcacggacacctatgaagaccagcagtggagaccccccaagcccactggtgaaacagctgagtgaagtatttgaaactgaagactctaaatcaaatcttcccccagagcctgttctgcccccagaggcacctttatcttctgaattggacttgcctctgggtacccagttatctgttgaggaacagatgccaccttggaaccagactgagttcccctccaaacaggtgttttccaaggaggaagcaagacagcccacagaaacccctgtggccagccagagctccgacaagccctcaagggaccctgagactcccagatcttcaggttctatgcgcaatagatggaaaccaaacagcagcaaggtactagggagatcccccctcaccatcctgcaggatgacaactcccctggcaccctgacactacgacagggtaagcggccttcacccctaagtgaaaatgttagtgaactaaaggaaggagccattcttggaactggacgacttctgaaaactggaggacgagcatgggagcaaggccaggaccatgacaaggaaaatcagcactttcccttggtggagagctaggccctgcatggccccagcaatgcagtcacccagggcctggtgatatctgtgtcctctcaccccttctttcccagggatactgaggaatggcttgttttcttagactcctcctcagctaccaaactgggactcacagctttattgggctttctttgtgtcttgtgtgtttcttttatattaaaggaagtaattttaaatgttactttaaaaaggtaaaaaaaaaaaaaaaaaaaaa

SEQ ID NO: 6 - DNA, 822 bp, Xenopus laevis
  misc_feature (720)..(720): wherein n is g, a, t or c
ccacgcgtccggcccgccgggccccgtccccagccccagtggagatgggttcgactcagagcgtctcaggcactccagcacggcctctgccacgcaacaagcaagtggctcgagtagcagaccctcgttcacctagtgctggcatccagcgcactcctattcaggtagagagctctccacagccaagcctaccagcagaacagctgaacggtctcaaacaggcacaagacccagatccccgctctcctactcttggcattgcacggacacccatgaagatcagtggtccagaccctcagtgctcactggtgaaagagctgagcgaagtattggagacagaagcgtcggaatcgatttcctccccagagcttgctctgccccgggaaacgcctttattttatgacctggacctgtcttcagatcctcagttatcccctgaggaccagttactgccttggagccaggctgaactcgatcccaaacaggtgtttaccaaggaggaagccaaacaatccgcagaaactatagctgccagccagaactcagacaagccctccagagacccagagactccccagtcctcaggttctaagcgcagcagacgaaaagcaaacagcaaggttctagggaggtcccctctcacatcctgcagatgacactcccctgggacttgacctacgacaggtagcggcctctgcctcatgagacgttaagactaangaagatcgtctgnactggagatctcagctgagagagccgagccacagacacacagaatcacttgctgtgaactagtggcgattccagccagagcttcatctc

SEQ ID NO: 7 - protein, 17 aa, Artificial Sequence
  synthetic peptide used as internal standard
NEGPQKGSPVSSWRTNN

(19) United States
(12) Patent Application Publication
(10) Pub. No.: US 2005/0130175 A1
(43) Pub. Date: Jun. 16, 2005

(54) Novel cell cycle genes required for mitotic entry

(75) Inventors: Nagi G. Ayad, Brookline, MA (US); Marc W. Kirschner, Newton, MA (US)

Correspondence Address: BANNER & WITCOFF, LTD., 28 STATE STREET, 28th FLOOR, BOSTON, MA, 02109-9601, US

(73) Assignee: President and fellows of Harvard College, Cambridge, MA (US)

(21) Appl. No.: 10/817,204

(22) Filed: Apr. 2, 2004

Related U.S. Application Data
(60) Provisional application No. 60/459,788, filed on Apr. 2, 2003.

(51) Int. Cl. C12Q 1/68; G01N 33/574; C07H 21/04; C12N 9/64

(52) U.S. Cl. 435/6; 435/69.1; 435/226; 435/320.1; 435/325; 536/23.2; 530/388.26; 435/7.23

(57) ABSTRACT

The invention provides nucleic acid molecules, designated Tome-1 nucleic acid molecules, which encode Tome-1, a protein that modulates mitotic entry. The invention also provides recombinant expression vectors containing Tome-1 nucleic acid molecules and host cells into which the expression vectors have been introduced. The invention still further provides Tome-1 proteins, fusion proteins, antigenic peptides and anti-Tome-1 antibodies. The invention also provides methods for the identification of modulators of Tome-1, methods of modulating Tome-1, methods of modulating mitosis, and methods for the treatment of disorders aberrant cell proliferation, such as cancer.

### RELATED APPLICATIONS

This application claims priority to U.S. Provisional Patent Application No. 60/459,788, filed on Apr. 2, 2003, hereby incorporated by reference in its entirety for all purposes.

### STATEMENT OF GOVERNMENT INTERESTS

This invention was made with government support under grant number R01GM 39023-16, awarded by the National Institutes of Health. The Government has certain rights in the invention.

### BACKGROUND

Progression through the eukaryotic cell cycle requires the coordinated activity of proteolytic triggers and kinase cascades (King et al. (1995) Cell 81:279). A key regulatory complex essential for coordinating the cell cycle in mitosis is the anaphase promoting complex or APC. The APC is an E3 ligase that mediates transfer of ubiquitin to critical cell cycle regulators such as cyclin B, securin and geminin, thereby targeting them for destruction by the 26S proteasome (Peters (1999) Exp. Cell Res. 248:339).

The timing of APC substrate recognition is precisely regulated during the cell cycle. APC only targets substrates for degradation during mitosis and G1 (Fang et al. (1998) Mol. Cell 2:163). For the APC to be active at these phases of the cell cycle, it must associate with one of two WD40 containing proteins, CDC20 or CDH1. CDC20 activates the APC during mitosis, while CDH1 binds the APC at the end of mitosis and throughout G1. CDC20 levels decrease dramatically during G1, since CDC20 itself is a substrate of the APC when activated by CDH1 (APC^{CDH1}) (Fang et al. (1998) Mol. Cell 2:163; Visintin et al. (1997) Science 278:460).

APC activity is also positively regulated by phosphorylation. For example, phosphorylation of the core APC subunits CDC27 and CDC16 is required for achieving maximal APC activity during mitosis (King et al. (1995) Cell 81:279; Kotani et al. (1999) J. Cell Biol. 146:791; Kramer et al. (2000) Mol. Biol. Cell 11:1555). One of the kinases responsible for phosphorylating the APC is cdk1/cyclin B, a kinase whose activity is exquisitely controlled by both proteolytic and phosphorylation mechanisms during the cell cycle (King et al. (1996) Science 274:1652; King et al. (1995) Cell 81:279). Cdk1 activation occurs when inhibitory phosphorylations (T14, Y15) are removed from cdkl by the phosphatase CDC25. CDC25 phosphatase activity and the opposing kinase activity of the inhibitory tyrosine kinase wee1 act as a switch (Gautier et al. (1991) Cell 67:197; Russell et al. (1987) Cell 49:559; Solomon et al. (1991) Cold Spring Herb. Symp. Quant. Biol. 56:427). When CDC25 activity is greater than wee1 activity, cdk1 becomes active, and the cell enters mitosis.

One way cells insure that CDC25 activity is greater than wee1 activity at the end of G2 is by degrading wee1 at this time. Recent studies have indicated that wee1 is degraded during G2 in a CDC34-dependent manner in Xenopus egg extracts (Michael et al. (1998) Science 282:1886). Since CDC34 is an E2 enzyme known to interact with the SCF ubiquitin ligase components Skp-1, Cul-1, Rbx, and the F box substrate receptor protein, wee1 degradation should require the activity of at least one SCF ligase (Peters (1998) Curr. Opin. Cell Biol. 10:759). However, the identity of the F box protein required for vertebrate wee1 degradation is unknown.

Vertebrate wee1 is thought to be part of the DNA replication checkpoint pathway (Michael et al. (1998) Science 282:1886). The control of wee1 degradation during the somatic cell cycle could provide a critical link between DNA replication and mitotic entry. Therefore, identification of factors essential for wee1 degradation is crucial for understanding both mitotic entry and the DNA replication checkpoint pathway.

### SUMMARY OF THE INVENTION

Studies in yeast, Xenopus and mammalian cells have clearly demonstrated that proteolysis and phosphorylation must work in an integrated fashion in order for cell division to occur faithfully or even at all. Central to this process are the regulated activities of E3 ubiquitin ligases and their substrates. Two of the best-studied E3 ligases active during the cell cycle are the APC and SCF ligases. Both ligases are similar in that they are multi-subunit complexes containing ring finger and cullin proteins. They differ completely in their specificity and the protein composition of the complex. Furthermore, the APC is only active after metaphase and during G1, while many SCF ligases target substrates for degradation during the S or G2 phases of the cell cycle.

A key SCF substrate degraded during the G2 phase of the cell cycle is the wee1 tyrosine kinase. Weel levels decrease as cells enter G2, thereby allowing for cdkl activation at the end of G2 (Michael et al. (1998) Science 282:1886). While wee1 degradation was not apparent in the early studies of the cell cycle in nucleus-free cytoplasmic extracts, recent studies have shown that it is important in somatic cells and very likely in embryonic systems as well. Before M phase, wee1 remains in the nucleus, possibly protecting the nucleus from cytoplasmically activated cdk1 (Heald et al. (1993). Cell 74:463). Since cdk1/cyclin B protein levels would have reached a level capable of activating mitosis, even a slight reduction in the amount of wee1 would tip the balance in favor of active cdk1 and allow for nuclear envelope breakdown and mitotic progression.

The present invention is based in part on the identification and characterization of the gene product Tome-1, which favors the active form of cdk1 by targeting wee1 for degradation. One important feature of this protein is that it is itself degraded. Without intending to be bound by theory, degradation of Tome-1 could insure that degradation of wee1 is transient and only occurs during G2. Furthermore, without intending to be bound by theory, it is also important that Tome-1 degradation is APC mediated, since this indicates that the activities of ubiquitinylation machineries are interdependent during the cell cycle.

The Tome-1 molecules of the present invention are useful as modulating agents to regulate mitosis. The Tome-1 nucleic acids and polypeptides of the present invention are useful for both in vitro and in vivo modulation of mitosis, as well as for the treatment of disorders associated with aberrant cellular proliferation such as cancer.

Accordingly, embodiments of the present invention are directed to nucleic acid molecules and polypeptides encoding Tome-1, i.e., Tome-1 nucleic acids, protein molecules and their analogs. In particular, the present invention is directed to methods of detecting nucleic acids and polypeptides that encode Tome-1 in samples, methods of modulating Tome-1 activity (e.g., modulating wee1 ubiquitinylation, wee1 degradation, and mitotic entry), and methods of identifying modulators of Tome-1 activity. The present invention also features Tome-1 nucleic acid molecules that specifically detect Tome-1 nucleic acid molecules relative to non-Tome-1 nucleic acid molecules.

Embodiments of the present invention also relate to vectors encoding Tome-1 nucleic acid molecules, such as recombinant expression vectors. Vectors encoding Tome-1 nucleic acids can be provided in host cells. Accordingly, the present invention provides methods for producing Tome-1 nucleic acids and polypeptides by culturing a host cell containing a recombinant expression vector in a suitable medium to produce Tome-1 nucleic acids and polypeptides.

The Tome-1 polypeptides of the present invention or biologically active portions thereof, can be operatively linked to a non-Tome-1 polypeptide (e.g., heterologous amino acid sequences) to form fusion proteins. Embodiments of the present invention further include antibodies, such as monoclonal or polyclonal antibodies, that specifically bind Tome-1 polypeptides of the invention. In addition, the Tome-1 polypeptides or biologically active portions thereof can be incorporated into pharmaceutical compositions, which optionally include pharmaceutically acceptable carriers.

Embodiments of the present invention further provide methods for modulating Tome-1 activity. Such methods include contacting a Tome-1 nucleic acid, a Tome-1 polypeptide, a cell capable of expressing a Tome-1 nucleic acid or polypeptide, or a subject, with an agent that modulates Tome-1 activity. Modulating Tome-1 with a compound can be useful for increasing or decreasing mitosis. Embodiments of the present invention also provide methods for treating a disorder in a subject by modulating Tome-1 activity. Compounds of the present invention can inhibit Tome-1 activity, or stimulate Tome-1 activity. Useful compounds include antibodies that specifically bind to a Tome-1 protein, compounds that increase or decrease expression of Tome-1 by modulating transcription of a Tome-1 gene or translation of a Tome-1 mRNA, and nucleic acid molecules having a nucleotide sequence that is antisense to the coding strand of a Tome-1 mRNA or a Tome-1 gene. Tome-1 modulators of the present invention can include Tome-1 polypeptides, Tome-1 nucleic acid molecules, peptides, peptidomimetics, or other small molecules.

It will be recognized by the person of ordinary skill in the art that the compounds, compositions, methods and kits disclosed herein provide significant advantages over prior technology. Compounds, compositions, methods and kits can be designed or selected to relieve and/or alleviate symptoms in a patient suffering from one or more disorders. These and other aspects and examples are described below. Other features and advantages of the invention will be apparent from the following detailed description and claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present invention will be more fully understood from the following detailed description of illustrative embodiments taken in conjunction with the accompanying drawings in which:

FIG. 1 depicts an alignment of mouse (SEQ ID NO:1), human (SEQ ID NO:2), and Xenopus (SEQ ID NO:3) Tome-1 protein. Identical residues are shaded, while similar residues are boxed.

FIGS. 2A-B depict autoradiographs of Tome-1 degradation. (A) shows Tome-1 degradation in Xenopus egg extracts is APC CDH1 dependent and requires active proteasomes and an intact KEN sequence. Xenopus Tome-1 was in vitro translated in a coupled transcription/translation system in the presence of ³⁵S-methionine and added to interphase extracts either containing or lacking CDH1. The degradation reactions were allowed to proceed for 0, 30, 60 or 90 minutes at room temperature, stopped by adding SDS sample buffer, and analyzed by SDS-PAGE. Top: autoradiograph showing degradation of wild-type Tome-1 in the presence or absence of CDH1 in Xenopus egg extracts. Middle: degradation of a mutant of Tome-1 containing a KEN to AAA mutation at residues 372-374. Bottom: degradation of wild-type Tome-1 in CDH1-supplemented extracts containing either 100 μM MG132 (left) or 100 μM N-cyclin B. (B) showsTome-1 is stable in a Δ90-extract. Top: in vitro translated Tome-1 was incubated with egg extracts driven into mitosis with Δ90-cyclin B, and the extent of degradation was measured at 0, 30, 60 or 90 minutes. Bottom: control indicating that securin is degraded in Δ90-cyclin B supplemented extract used in top panel.

FIGS. 3A-3B depict Tome-1 degradation. (A) shows endogenous Tome-1 is degraded during G1. Graphic representation of the percentage of HeLa cells in the G2/M, S or G1 phases of the cell cycle after culturing cells in the absence of thymidine for the indicated times. (B) shows anti-Tome-1 or Anti-cyclin B immunoblot of SDS lysates of cells taken at the various time points in part A. Asterisk indicates position of a nonspecific band present after cyclin B Western.

FIGS. 4A-4B depict immunofluorescence of cells expressing Tome-1. (A) shows Immunofluorescence of CFPAC cells stained with either anti-Tome-1 antibody (red) or Dapi (blue) in a mitotic cell (top) and an interphase cell (bottom). (B) shows NIH-3T3 fibroblasts were transfected with myc-tagged Tome-1 and stained with anti-myc antibodies.

FIGS. 5A-5D depict Tome-1 association with Skp-1. (A) shows Tome-1 co-purifies with Skp-1. XTC cell extracts were first bound to an anion-exchange column (Hi-Trap Q) and eluted using a linear NaCl gradient. Peak fractions containing both Tome-1 and Skp-1, as judged by anti-Tome-1 or anti-Skp-1 immunoblots, were further analyzed on a Smart System Superose 6 Column. Fractions from the Superose 6 column were assayed for Tome-1 or Skp-1 immuno-reactivity as shown. Position of molecular weights markers (660, 440 and 220 kDa) are indicated. (B) shows that Tome-1 associates with Skp-1 in vitro. Autoradiograph demonstrating results of glutathione precipitation assay using ³⁵S-labeled-Skp-1 and GST-Tome-1 or an equivalent amount of GST (left) or various mutants of Tome-1 (right). (C) shows that Myc-Tome-1 interacts with HA-tagged Skp-1 and Flag-Cul-1. Myc-Tome- 1, Myc-Tome-1 AFbox, or Myc-CR16 were co-transfected with HA-Skp-1 and Flag-Cul-1, and the extent of the respective interactions determined by anti-myc immunoprecipitations. Western blots to detect myc, HA, or Flag are provided. Single and double asterisks indicate the position of IgG heavy and light chains, respectively. (D) shows Xenopus Tome-1 associates with HA-Cul-1 and Skp-1 in vitro. Recombinant Xenopus Tome-1 was incubated with extracts of SF9 cells expressing HA-Cul-1 and Skp-1 and immunoprecipitated with anti-HA beads. The identity of the bands labeled Tm1, Skp-1, and HA-Cul-1 was confirmed by LC-MS/MS. Single and double asterisks indicate the position of IgG heavy and light chains, respectively.

FIGS. 6A-6D depict ΔN-Tome-1 Inhibition of mitotic entry. (A) shows ΔN-Tome-1 injection into embryos inhibits mitotic progression. 3 nl of ΔN-Tome-1 (0.5 mg/ml) was injected into both sides of Xenopus embryos at the two-cell stage (bottom), and the embryos were filmed along with uninjected embryos (top). (B) shows ΔN-Tome-1 inhibits mitotic entry in extracts. Tome-1 or ΔN-Tome-1 was included in extracts cycling between interphase and mitosis. Nuclear morphology of added sperm nuclei was assayed at 60 or 120 minutes. (C) shows ΔN-Tome-1 increases tyrosine phosphorylation on cdkl. Immunoblot analysis of extracts shown in Part B for tyrosine phosphorylation on cdk1 at 0, 60 or 120 minutes after Ca²⁺ addition. “Control” lanes correspond to buffer-only control. (D) shows ΔN-Tome-1 does not inhibit mitotic entry in wee1-depleted extracts. Left: wee1-depleted extracts (−wee1) or mock-depleted extracts (+wee1) were supplemented with ΔN-Tome-1 and the percent of nuclear envelope breakdown (% NEB) calculated after microscopic analysis of nuclei. Right: nuclear envelope breakdown was quantified in egg extracts where wee1 was depleted (−wee1) or not depleted (+wee1).

FIGS. 7A-7C depict Tome-1 is required for mitotic entry and wee1 degradation. (A) shows immunodepletion of Tome-1 delays entry into mitosis. CSF extracts were depleted with either anti-Tome-1 antibody or anti-IgG control antibody and released into interphase. Immunoblot for Tome-1 (left) shows amount of Tome-1 remaining in each of the depleted extracts. Right: H1 kinase assays were performed for samples taken after calcium release (0 time point is 20 minutes after Ca²⁺ release). (B) shows immunodepletion of Tome-1 inhibits wee1 degradation. CSF extracts were depleted as in Part A and released with calcium for 20 minutes, at which time ³⁵S-labeled-wee1 was added. Aliquots were taken after 0, 30, 60, 120 or 150 minutes of incubation at room temperature, and analyzed by SDS-PAGE and autoradiography (Left). Right: quantification of the amount of wee1 remaining presented in left panel. (C) shows immunodepletion of Tome-1 does not affect β-catenin degradation. Autoradiograph demonstrating the extent of ³⁵S-labeled β-catenin degradation in a Tome-1-depleted or mock-depleted extract, as in Part B.

FIGS. 8A-8F depict Tome-1 is required for mitotic entry and wee1 degradation. (A) shows degradation of the nuclear pool of wee1 requires Tome-1. Autoradiograph showing extent of ₃₅S-labeled wee1 degradation observed in a Tome-1-depleted (Tm1 Dep.) or mock-depleted (Mock Dep.) extract after isolating nuclei. (B) shows expression of ΔN-Tome-1 inhibits wee1 degradation in 293 cells. An inducible cell line was generated expressing ΔN-Tome-1. An immunoblot for endogenous wee1 or APC2. (C) shows a Myc-tag immunoblot for induced (I) or uninduced (UI) expression of ΔN-Tome-1 in 293 cells arrested with nocodazole for 12 hours. (D) shows Tome-1 is required for wee1 degradation in somatic cells. 293 cells were transfected with small interfering RNA targeting endogenous Tome-1, and the extent of wee1 degradation assayed after pulse-chase analysis using an anti-wee1 antibody. An autoradiograph of endogenous wee1 immunoprecipitated from the indicated transfected cells 0, 60 or 120 minutes after pulse with ³⁵S-methionine and cysteine. (E) shows Tome-1 and APC2 Western blot analysis of cells transfected with either oligo1, oligo2, or oligo3. (F) shows quantification of the amount of wee1 in Part E. The cell cycle profile of all three transfected cell lines tested by pulse-chase analysis was analyzed by FACS analysis and found to be identical.

FIGS. 9A-9D depict Tome-1 interacts with phosphorylated wee1 and mediates its ubiquitinylation. (A) shows Tome-1 addition enhances ubiquitinylation of wee1 in Xenopus egg extracts. Left: in vitro translated, ³⁵S-labeled wee1 was added to Xenopus egg extracts in the presence or absence of recombinant Tome-1 (+/−Tome-1), GST-ubiquitin (GST-Ub), or methyl-ubiquitin (methylUb). (B) shows quantification of wee1 ubiquitinylation shown in Part A. (C) shows wild-type, but not a serine mutant of wee1 at position 38, interacts with GST-Tome-1. In vitro translated wild-type or S38A wee1 were incubated in nuclei containing Xenopus interphase egg extracts supplemented with GST-Tome-1 (lanes 1-6) or as a control, a GST fusion with the C terminus of the actin nucleating scaffolding protein N-WASP, GST-VCA (lanes 7 and 8). The amount of wild-type-wee1 or S38A that interacted with GST-Tome-1 was determined by wee1 Western analysis performed on glutathione precipitated samples (lanes 1-3 for wild-type wee1 and lanes 4-6 for S38A wee1 ). Lanes 9 and 10 indicate 5% of input wee1 or S38A wee1 used in binding assays. (D) shows wild-type-wee1 , but not a S38A mutant of wee1 , is degraded in Xenopus egg extracts. Autoradiograph demonstrating extent of degradation of ³⁵S-labeled wild-type or S38A wee1 in Xenopus egg extracts.

FIGS. 10A-10D depict Tome-1 interacts with phosphorylated wee1 and mediates its ubiquitinylation. (A) shows injection of S38A wee1 into Xenopus embryos inhibits mitotic entry. In vitro transcribed RNAs encoding the wild-type and mutant forms of wee1 were made with mMessage mMachine (Ambion). The RNA was diluted to 0.5 ng/nl, and 2 ng was injected into two- to four-cell embryos. The embryos were collected at stage 9-9.5 and fixed in formalin. The embryos shown are representative of 21 WT, 25 S38A, and 15 KM-wee1 -injected embryos. (B) shows S38A wee1 is stable in vivo. Five embryos were also taken for immunoblot analyses. The exogenous, flag-tagged wee1 was detected with an anti-flag Ab (M2, Sigma). An agarose gel indicates that an equivalent amount of wee1 RNA was injected into each of the embryos. (C) shows extracted ion current trace of endogenous phosphorylated and unphosphorylated wee1 peptides and the respective synthesized internal standards. (D) shows phosphorylation of serine 38 decreases in response to the replication checkpoint. Heavy indicates 13C-modified valine (underlined) containing peptide was used as an internal standard. Quantification of phosphorylated and unphosphorylated endogenous wee1 peptides present in egg extracts, egg extracts supplemented with nuclei in the presence, or absence of aphidicolin was performed as described in Stemmann et al. ((2001) Cell 107:715).

FIG. 11 depicts a model of Tome-1 activity during the cell cycle. Tome-1 elicits mitotic entry by decreasing wee1 levels, thereby tipping the balance to active cdk1.

FIG. 12 depicts Tome-1 does not affect wee1 activity. An in vitro kinase assay was performed in the presence or absence of recombinant Tome-1. Purified cdk1/cylin B complex (NEB) was incubated in the presence (lanes 1 and 4) or absence (lanes 2, 3, 5 and 6) of in vitro translated wee1 for 30 minutes at 30° C. Recombinant Tome-1 (100 nM) was added to reactions four to six. Phospho-tyrosine (Y) cdk1 Western analysis was performed on all samples to determine the extent of Wee1 activity. Including Tome-1 in the reaction had no effect on Wee1 activity since wee1 was able to phosphorylate cdk1 in the presence of Tome-1 (cdk1 Western was included as loading control).

FIG. 13 depicts the cDNA sequence encoding murine Tome-1 (SEQ ID NO:4).

FIG. 14 depicts the cDNA sequence encoding human Tome-1 (SEQ ID NO:5).

FIG. 15 depicts the cDNA sequence encoding Xenopus Tome-1 (SEQ ID NO:6).

### DETAILED DESCRIPTION OF CERTAIN EXAMPLES

Entry into mitosis requires the activation of cdk1/cyclin B, while mitotic exit is achieved when the same kinase activity decreases, as cyclin B is degraded. Cyclin B proteolysis is mediated by the anaphase promoting complex (APC), an E3 ligase that is active at anaphase in mitosis through G1. Embodiments of the present invention relate to the isolation and characterization of a G1 substrate of the APC, referred to herein as “Tome-1” for trigger of mitotic entry. Tome-1 is a cytosolic protein required for proper activation of cdk1 /cyclin B and mitotic entry. Tome-1 associates with Skp-1 and is required for degradation of the cdk1 inhibitory tyrosine kinase wee1. Without intending to be bound by theory, Tome-1 acts as part of an SCF-type E3 for wee1. Degradation of Tome-1 during GI allows for wee1 accumulation during interphase, thereby providing a critical link between the APC and SCF pathways in regulation of cdk1/cyclin B activity and thus mitotic entry and exit. Embodiments of the present invention are thus directed to the regulation of Tome-1 for the temporal control of mitosis.

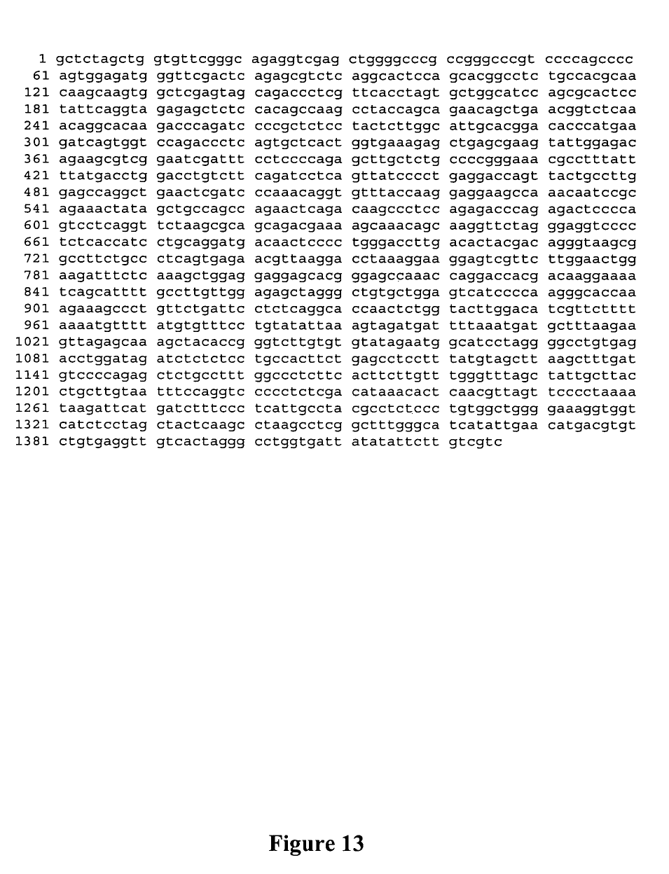

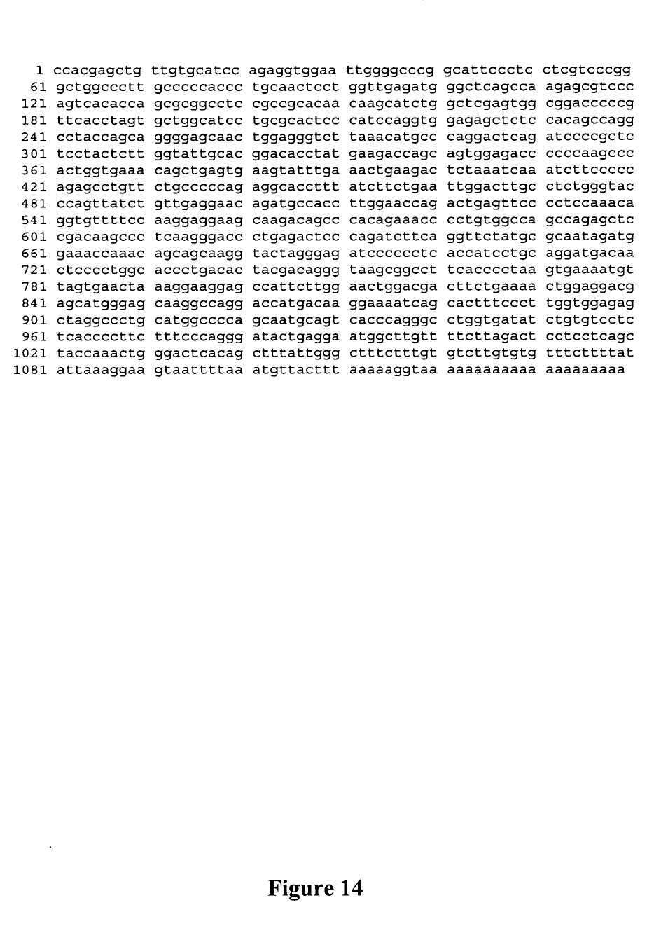

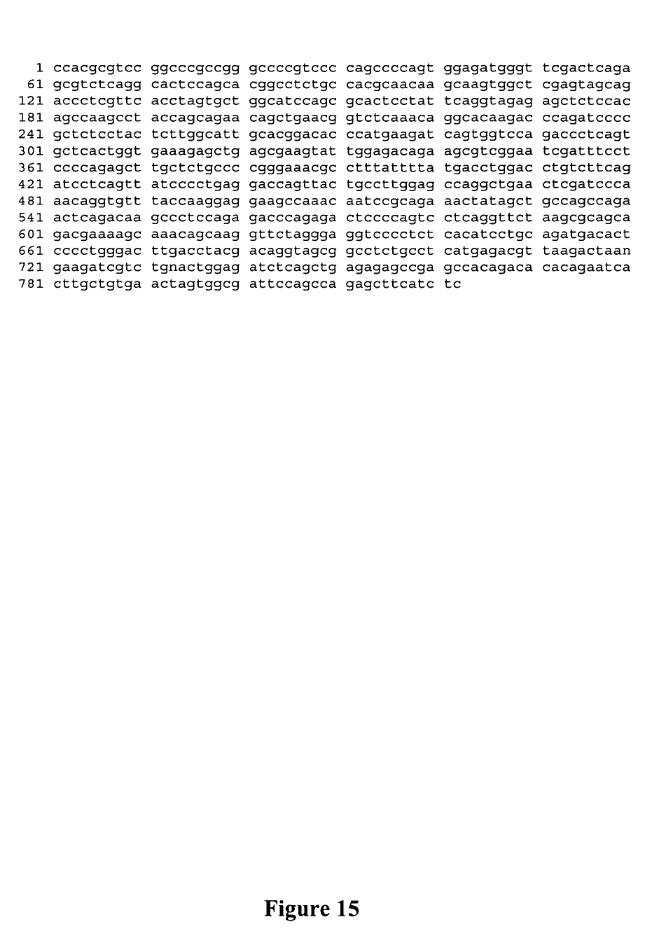

The Tome-1 cDNA sequences (FIGS. 13-15, SEQ ID NOs:4, 5 and 6) encode Tome-1 proteins (SEQ ID NOs:1, 2 and 3) that contain two amino-terminal destruction boxes, an F box and a carboxy-terminal KEN sequence. The Homo sapiens Tome-1 cDNA sequence is set forth in FIG. 14 and SEQ ID NO:5, and the protein sequence is set forth as SEQ ID NO:2. The Mus musculus Tome-1 cDNA sequence is set forth in FIG. 13 and SEQ ID NO:4, and the protein sequence is set forth as SEQ ID NO:1. The Xenopus laevis Tome-1 cDNA sequence is set forth in FIG. 15 and SEQ ID NO:6, and the protein sequence is set forth as SEQ ID NO:3.

As used herein, the term “destruction box” refers to an amino acid motif that directs proteolysis of the protein containing the destruction box by ubiquitin and anaphase promoting complex (APC). Destruction boxes are described further in Yamano et al. ((1998) EMBO J. 17:5670), incorporated herein by reference in its entirety. As used herein, the term “F box” refers to an amino acid motif that allows protein-protein interactions and is sometimes associated with proteins that are critical for the controlled degradation of cellular regulatory proteins. F boxes are described further in Cenciarelli et al. (1999) Curr. Biol. 9:1177, and Kipreos et al. (2000) Gen. Biol. 1:3002.1, incorporated herein by reference in their entirety. As used herein, the terms “KEN sequence” and “KEN box” refer to a KEN amino acid motif that directs ubiquitination and subsequent degradation of the protein containing the KEN motif. KEN boxes are described further in Pfleger et al. ((2000) Genes Dev. 14:655), incorporated herein by reference in its entirety.

In one embodiment, a Tome-1 nucleic acid molecule of the invention is at least 40%, 50%, 60%, 65%, 70%, 75%, 80%, 85%, 86%, 87%, 88%, 89%, 90%, 91%, 92%, 93%, 94%, 95%, 96%, 97%, 98%, 98.9%, 99.0%, 99.1%, 99.2%, 99.3%, 99.4%, 99.5%, 99.6%, 99.7%, 99.8%, 99.9% or more identical to the nucleotide sequence (e.g., to the entire length of the nucleotide sequence) shown in SEQ ID NOs:4, 5 or 6.

In another embodiment, the nucleic acid molecule includes the nucleotide sequence shown in SEQ ID NOs:4, 5 or 6, or complements and/or analogs thereof.

In another embodiment, a Tome-1 nucleic acid molecule includes a nucleotide sequence encoding a protein having an amino acid sequence sufficiently identical to the amino acid sequence of SEQ ID NOs:1, 2 or 3. In another embodiment, a Tome-1 nucleic acid molecule includes a nucleotide sequence encoding a protein having an amino acid sequence at least 40%, 50%, 60%, 65%, 70%, 75%, 80%, 85%, 86%, 87%, 88%, 89%, 90%, 91%, 92%, 93%, 94%, 95%, 96%, 97%, 98%, 99% or more identical to the amino acid sequence of SEQ ID NOs:1, 2 or 3.

The present invention also features nucleic acid molecules, such as Tome-1 nucleic acid molecules and analogs thereof, which specifically detect Tome-1 nucleic acid molecules relative to nucleic acid molecules encoding non-Tome-1 proteins. For example, in one embodiment, such a nucleic acid molecule is at least 15, 30, 50, 100, 250, 300, 350, 400, 450, 500, 550, 600, 650, 700, 750, 760, 770, 780, 790, 800, 810, 820, 830, 840, 850, 900, 950, 960, 970, 980, 990, 1000, 1010, 1020, 1030, 1040, 1050, 1100, 1150, 1200, 1250, 1300, 1350, 1400, 1410, 1420, 1430, 1440, 1450 or more nucleotides in length and hybridizes, preferably under stringent conditions, to a nucleic acid molecule comprising the nucleotide sequence shown in SEQ ID NOs:4, 5 or 6.

In other embodiments, the nucleic acid molecule encodes a naturally occurring allelic variant of a polypeptide comprising the amino acid sequence of SEQ ID NOs: 1, 2 or 3, wherein the nucleic acid molecule hybridizes to a nucleic acid molecule comprising SEQ ID NOs:4, 5 or 6 under stringent conditions.

As used herein, the term “hybridizes under stringent conditions” is intended to describe conditions for hybridization and washing under which nucleotide sequences at least 60% identical to each other typically remain hybridized to each other. In one embodiment, the conditions are such that sequences at least about 70%, at least about 80%, even at least about 85% or 90% identical to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6X sodium chloride/sodium citrate (SSC) at about 45° C., followed by one or more washes in 0.2×SSC, 0.1% SDS at 50° C., preferably at 55° C., and more preferably at 60° C. or 65° C. Preferably, a nucleic acid molecule of the invention that hybridizes under stringent conditions to the sequence of SEQ ID NO:2 corresponds to a naturally-occurring nucleic acid molecule. As used herein, the term “analog” includes an RNA or DNA molecule that can be identified using these stringent hybridization conditions as well as amino acids and polypeptides encoded by the RNA or DNA so identified. As used herein, a “naturally-occurring” nucleic acid molecule refers to an RNA or DNA molecule having a nucleotide sequence that occurs in nature (e.g., encodes a natural protein).

Another embodiment of the invention provides a nucleic acid molecule which is antisense to a Tome-1 nucleic acid molecule, e.g., the coding strand of a Tome-1 nucleic acid molecule.

Another embodiment of the invention features isolated or recombinant Tome-1 proteins and polypeptides. In one embodiment, Tome-1 includes at least one destruction box, and more preferably two destruction boxes. In another embodiment, Tome-1 contains at least one F box. In another embodiment, Tome-1 includes at least one carboxyl-terminal KEN sequence. In one embodiment, a Tome-1 polypeptide or Tome-1 analog includes at least one destruction box, one F box and one carboxyl-terminal KEN sequence and has an amino acid sequence at least about 40%, 50%, 60%, 65%, 70%, 75%, 80%, 85%, 86%, 87%, 88%, 89%, 90%, 91%, 92%, 93%, 94%, 95%, 96%, 97%, 98%, 99% or more identical to the amino acid sequence of SEQ ID NOs:1, 2 or 3, or the amino acid sequence encoded by a nucleic acid molecule having the nucleic acid sequence of SEQ ID NOs:4, 5 or 6. In another embodiment, Tome-1 modulates mitotic entry. In yet another embodiment, Tome-1 includes at least one F box and is encoded by a nucleic acid molecule having a nucleotide sequence which hybridizes under stringent hybridization conditions to a nucleic acid molecule comprising the nucleotide sequence of SEQ ID NOs:4, 5 or 6.

In another embodiment, the invention features fragments of the proteins having the amino acid sequence of SEQ ID NOs: 1, 2 or 3, wherein the fragment comprises at least 10 amino acids (e.g., contiguous amino acids) of the amino acid sequence of SEQ ID NOs:1, 2 or 3. In another embodiment, Tome-1 has the amino acid sequence of SEQ ID NOs:1, 2 or 3.

In another embodiment, the invention features an isolated Tome-1 which is encoded by a nucleic acid molecule having a nucleotide sequence at least about 40%, 50%, 60%, 65%, 70%, 75%, 80%, 85%, 86%, 87%, 88%, 89%, 90%, 91%, 92%, 93%, 94%, 95%, 96%, 97%, 98%, 98.9%, 99.0%, 99.1%, 99.2%, 99.3%, 99.4%, 99.5%, 99.6%, 99.7%, 99.8%, 99.9% or more identical to a nucleotide sequence of SEQ ID NOs:4, 5 or 6, or a complement thereof.

As used herein, a “Tome-1 activity,” “biological activity of Tome-1 ,” or “functional activity of Tome-1 ,” refers to an activity exerted by a Tome-1 protein, polypeptide or nucleic acid molecule on, for example, a Tome-1-responsive cell or on a Tome-1 substrate (e.g., wee1) as determined in vivo or in vitro. In one embodiment, a Tome-1 activity is a direct activity, such as association with a Tome-1 target molecule. A “target molecule” or “binding partner” of Tome-1 is a molecule with which Tome-1 binds or interacts in nature (e.g., wee1 , Skp-1, Cul-1 and the like). A Tome-1 activity can also be an indirect activity, such mitotic entry mediated by interaction of Tome-1 with a Tome-1 target molecule or release from mitosis mediated by Tome-1 degradation.

The Tome-1 proteins of the present invention can have one or more of the following activities: (1) modulating ubiquitinylation of wee1 ; (2) modulating degradation of wee1 (3) modulating SCF complex components (e.g., Skp-1, Cul-1 and the like); (4) modulating entry of a cell into the cell cycle; (5) modulating progression of a cell through the cell cycle; (6) modulating release of a cell from the cell cycle; (7) modulating cell growth; (8) modulating cellular proliferation; (9) modulating tumorigenesis; and (10) modulating mitogenesis.

As used herein, the term “modulate” refers to a stimulation or inhibition of an activity, such as regulation of wee1 ubiquitinylation, wee1 degradation, Tome-1 degradation, cdk1/cyclinB phosphorylation and/or dephosphorylation, mitotic entry and the like. As used herein, the term “ubiquitinylation” refers to the formation of a peptide bond between ubiquitin and the side-chain NH₂ of a lysine residue in a target protein. Additional ubiquitin molecules are added, forming a multi-ubiquitin chain. The multi-ubiquitin chain is thought to direct the tagged protein to the proteasome for degradation. For a review of ubiquitination, see Chapter 3, Lodish et al., Molecular Cell Biology, (W.H Freeman & Co., N.Y., 2000), incorporated herein by reference in its entirety. As used herein, the term “degradation” refers to the proteolytic cleavage of a polypeptide at one or more cleavage site. Degradation may occur via the ubiquitin-mediated proteolytic pathway following, for example, ubiquitinylation of a polypeptide. As used herein, the terms “phosphorylation” and “dephosphorylation” refer to the addition or removal, respectively, of phosphate groups from serine, threonine, or tyrosine residues of a polypeptide as mechanism for regulating protein activity. Kinases catalyze phosphorylation, and phosphatases catalyze dephosphorylation. As used herein, the term “mitotic entry” refers beginning the process of cell division (i.e., the mitosis phase of the cell cycle). For a review of mitosis and the cell cycle, see Chapter 13, Lodish et al., Molecular Cell Biology, (W.H Freeman & Co., N.Y., 2000), incorporated herein by reference in its entirety.

As used herein, the terms “inhibit” and “inhibition” refer to a partial inhibition or a complete inhibition of an activity, such as an inhibition of a Tome-1 activity such that, for example, inhibition of wee1 degradation, inhibition of wee1 phosphorylation, inhibition of mitotic entry or the like results, or an inhibition of a disorder, disease, or condition such that therapeutic treatment and/or prophylaxis results. An inhibition of Tome-1 activity occurs, for example, when a cell expressing Tome-1 is contacted with a compound that inhibits and has a lower level of Tome-1 activity (i.e., the activities described herein) as compared to a cell expressing Tome-1 that is not contacted with the compound. A complete inhibition occurs, for example, when no mitosis or wee1 ubiquitination is observed when Tome-1 is contacted with the compound as compared to when Tome-1 is not contacted with the compound. A partial inhibition of Tome-1 activity occurs, for example, when mitosis or wee1 ubiquitination is observed in the presence of a compound, but at lower levels than in the absence of the compound. For example, one or more Tome-1 activities may be reduced to 99%, 95%, 90%, 85%, 80%, 75%, 70%, 65%, 60%, 55%, 50%, 45%, 40%, 35%, 30%, 25%, 20%, 15%, 10%, 5%, or 1% of the level of a Tome-1 activity in the absence of the compound.

As used herein, the terms “stimulate” and “stimulation” refer to an increase in a Tome-1 activity, such as an increase of wee1 degradation, wee1 ubiquitinylation, or mitosis, or a worsening of a disorder, disease, or condition. A stimulation in mitosis or wee1 stability is observed, for example, when a cell expressing Tome-1 is contacted with a compound that stimulates and has a higher level of Tome-1 activity as compared to a cell expressing Tome-1 that is not contacted with a compound. A stimulation in Tome-1 activity occurs, for example, when one or more Tome-1 activities may be observed at least at 101%, 102%, 103%, 104%, 105%, 106%, 107%, 108%, 109%, 110%, 115%, 120%, 125%, 130%, 135%, 140%, 145%, 150%, 160%, 170%, 180%, 185%, 190%, 200%, 250%, 300%, 350%, 400%, 500%, 600%, 700%, 800%, 900%, or higher levels when compared to the levels of Tome-1 activity observed in a cell not contacted with a compound that stimulates Tome-1 activity.

In at least certain examples, compounds described herein can be used in the treatment disorders associated with aberrant cellular proliferation (e.g., cellular proliferative disorders such as cancer). Treatment of cellular proliferative disorders is intended to include inhibition of proliferation including rapid proliferation. As used herein, the term “cellular proliferative disorder” includes disorders characterized by undesirable or inappropriate proliferation of one or more subset(s) of cells in a multicellular organism. The term “cancer” refers to various types of malignant neoplasms, most of which can invade surrounding tissues, and may metastasize to different sites (see, for example, PDR Medical Dictionary 1st edition (1995)). The terms “neoplasm” and “tumor” refer to an abnormal tissue that grows by cellular proliferation more rapidly than normal and continues to grow after the stimuli that initiated proliferation is removed (see, for example, PDR Medical Dictionary 1st edition (1995)). Such abnormal tissue shows partial or complete lack of structural organization and functional coordination with the normal tissue which may be either benign (i.e., benign tumor) or malignant (i.e., malignant tumor).

The language “treatment of cellular proliferative disorders” is intended to include the prevention of the growth of neoplasms in a subject or a reduction in the growth of pre-existing neoplasms in a subject. The inhibition also can be the inhibition of the metastasis of a neoplasm from one site to another. The neoplasms are preferably sensitive to one or more compounds that modulate Tome-1. Examples of the types of neoplasms intended to be encompassed by the present invention include but are not limited to those neoplasms associated with cancers of the breast, skin, bone, prostate, ovaries, uterus, cervix, liver, lung, brain, larynx, gallbladder, pancreas, rectum, parathyroid, thyroid, adrenal gland, immune system, neural tissue, head and neck, colon, stomach, bronchi, and/or kidneys.

Cellular proliferation disorders can further include disorders associated with hyperproliferation of vascular smooth muscle cells such as proliferative cardiovascular disorders, e.g., atheroscelrosis and restinosis. Cellular proliferation disorders can also include disorders such as proliferative skin disorders, e.g., X-linked ichthyosis, psoriasis, atopic dermatitis, allergic contact dermatitis, epidermolytic hyperkeratosis, and seborrheic dernatitis. Cellular proliferation disorders can further include disorders such as autosomal dominant polycystic kidney disease (ADPKD), mastocystosis, and cellular proliferation disorders caused by infectious agents such as viruses.

Screening Assays

The present invention provides a method (also referred to herein as a “screening assay”) for identifying modulators, i.e., candidate or test compounds or agents (e.g., peptides, cyclic peptides, peptidomimetics, small molecules, small organic molecules, or other drugs) which bind to Tome-1 proteins, have a stimulatory or inhibitory effect on, for example, Tome-1 expression, Tome-1 degradation or Tome-1 activity, or have a stimulatory or inhibitory effect on, for example, the expression or activity of Tome-1 substrate (e.g., wee1).

As used herein, the term “small organic molecule” refers to an organic molecule, either naturally occurring or synthetic, that has a molecular weight of more than about 25 daltons and less than about 3000 daltons, preferably less than about 2500 daltons, more preferably less than about 2000 daltons, preferably between about 100 to about 1000 daltons, more preferably between about 200 to about 500 daltons.

In one embodiment, the invention provides assays for screening candidate or test compounds which are substrates of Tome-1 or a Tome-1 polypeptide or biologically active portion thereof. In another embodiment, the invention provides assays for screening candidate or test compounds which bind to or modulate the activity of Tome-1 or a Tome-1 polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412), or on beads (Lam (1991) Nature 354:82), chips (Fodor (1993) Nature 364:555), bacteria (Ladner U.S. Pat. No. 5,223,409), spores (Ladner U.S. Pat. No. 5,223,409), plasmids (Cull et al. (1992) Proc. Natl. Acad. Sci. USA 89:1865) or on phage (Scott and Smith (1990) Science 249:386); (Devlin (1990) Science 249:404); (Cwirla et al. (1990) Proc. Natl. Acad. Sci. USA 87:6378); (Felici (1991) J. Mol. Biol. 222:301); (Ladner supra).

Examples of methods for introducing a molecular library of randomized nucleic acids into a population of cells can be found in the art, for example in U.S. Pat. No. 6,365,344, incorporated herein in its entirety by reference. A molecular library of randomized nucleic acids can provide for the direct selection of candidate or test compounds with desired phenotypic effects. The general method can involve, for instance, expressing a molecular library of randomized nucleic acids in a plurality of cells, each of the nucleic acids comprising a different nucleotide sequence, screening for a cell of exhibiting a changed physiology in response to the presence in the cell of a candidate or test compound, and detecting and isolating the cell and/or candidate or test compound.

In one embodiment, the introduced nucleic acids are randomized and expressed in the cells as a library of isolated randomized expression products, which may be nucleic acids, such as mRNA, antisense RNA, siRNA, ribozyme components, etc., or peptides (e.g., cyclic peptides). The library should provide a sufficiently structurally diverse population of randomized expression products to effect a probabilistically sufficient range of cellular responses to provide one or more cells exhibiting a desired response. Generally at least 10⁶, at least 10⁷, at least 10⁸, or at least 10⁹ different expression products are simultaneously analyzed in the subject methods. In one aspect methods maximize library size and diversity.

The introduced nucleic acids and resultant expression products are randomized, meaning that each nucleic acid and peptide consists of essentially random nucleotides and amino acids, respectively. The library may be fully random or biased, e.g., in nucleotide/residue frequency generally or per position. In other embodiments, the nucleotides or residues are randomized within a defined class, e.g. of hydrophobic amino acids, of purines, etc.

Functional and structural isolation of the randomized expression products may be accomplished by providing free (not covalently coupled) expression product, though in some situations, the expression product may be coupled to a functional group or fusion partner, preferably a heterologous (to the host cell) or synthetic (not native to any cell) functional group or fusion partner. Exemplary groups or partners include, but are not limited to, signal sequences capable of constitutively localizing the expression product to a predetermined subcellular locale such as the Golgi, endoplasmic reticulum, nucleoli, nucleus, nuclear membrane, mitochondria, chloroplast, secretory vesicles, lysosome, and the like; binding sequences capable of binding the expression product to a predetermined protein while retaining bioactivity of the expression product; sequences signaling selective degradation, of itself or co-bound proteins; and secretory and membrane-anchoring signals.

It may also be desirable to provide a partner which conformationally restricts the randomized expression product to more specifically define the number of structural conformations available to the cell. For example, such a partner may be a synthetic presentation structure: an artificial polypeptide capable of intracellularly presenting a randomized peptide as a conformation-restricted domain. Generally such presentation structures comprise a first portion joined to the N-terminal end of the randomized peptide, and a second portion joined to the C-terminal end of the peptide. Preferred presentation structures maximize accessibility to the peptide by presenting it on an exterior loop, for example of coiled-coils, (Myszka and Chaiken (1994) Biochemistry 33:2362). To increase the functional isolation of the randomized expression product, the presentation structures are selected or designed to have minimal biologically active as expressed in the target cell. In addition, the presentation structures may be modified, randomized, and/or matured to alter the presentation orientation of the randomized expression product. For example, determinants at the base of the loop may be modified to slightly modify the internal loop peptide tertiary structure, while maintaining the absolute amino acid identity. Other presentation structures include zinc-finger domains, loops on beta-sheet turns and coiled-coil stem structures in which non-critical residues are randomized; loop structures held together by cysteine bridges, cyclic peptides, etc.

In another embodiment, the present invention provides cyclic peptides for use in the libraries described herein. As used herein, the term “cyclic peptide” refers to a peptide configured in a loop. Cyclic peptides can be produced by generating a nucleotide sequence encoding a peptide to be cyclized flanked on one end with a nucleotide sequence encoding the carboxy-terminal portion of a split (or trans) intein (C-intein or I_{C}) and on the other end with a nucleotide sequence encoding the amino-terminal portion of a split intein (N-intein or I_{N}). Expression of the construct within a host system, such as bacteria or eukaryotic cells described herein, results in the production of a fusion protein. The two split intein compounds (i.e., I_{C} and I_{N}) of the fusion protein then assemble to form an active enzyme that splices the amino and carboxy termini together to generate a backbone cyclic peptide. Cyclic polypeptides can be generated using a variety of inteins. Methods of generating cyclic proteins can be found in the art, for example, in WO 00/36093 and WO 01/57183, incorporated herein by reference in their entirety.

As used herein, the term “intein” refers to a naturally-occurring or artificially-constructed polypeptide embedded within a precursor protein that can catalyze a splicing reaction during post-translation processing of the protein.

In one embodiment, an assay is a cell-based assay in which a cell which expresses a Tome-1 protein or biologically active portion thereof is contacted with a test compound and the ability of the test compound to modulate Tome-1 activity, e.g., mitotic entry, wee1 degradation and/or binding to Skp-1 and/or Cul-1, is determined. Determining the ability of the test compound to modulate Tome-1 activity can be accomplished by monitoring, for example, the ubiquitinylation or degradation of wee1 or mitotic entry or exit. Determining the ability of the test compound to modulate the ability of Tome-1 to bind to a substrate can be accomplished, for example, by coupling the Tome-1 substrate with a radioisotope or enzymatic label such that binding of the Tome-1 substrate to Tome-1 can be determined by detecting the labeled Tome-1 substrate in a complex. For example, compounds (e.g., Tome-1 substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., a Tome-1 substrate) to interact with Tome-1 without the labeling of any of the interactants. For example, a microphysiometer can be used to detect the interaction of a compound with Tome-1 without the labeling of either the compound or Tome-1 (McConnell, H. M. et al. (1992) Science 257:1906). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and Tome-1.

In another embodiment, an assay is a cell-based assay comprising contacting a cell expressing a Tome-1 target molecule (e.g., wee1) with a test compound and determining the ability of the test compound to modulate the activity of the Tome-1 target molecule. Determining the ability of the test compound to modulate the activity of a Tome-1 target molecule can be accomplished, for example, by determining the ability of the Tome-1 protein to bind to or interact with the Tome-1 target molecule, e.g., wee1 , Skp-1, Cul-1 or the like.

Determining the ability of Tome-1 or a biologically active fragment thereof, to bind to or interact with a Tome-1 target molecule can be accomplished by one of the methods described above for determining direct binding. In one embodiment, determining the ability of Tome-1 to bind to or interact with a Tome-1 target molecule can be accomplished by determining the activity of the target molecule. For example, the activity of the target molecule can be determined by detecting induction of a cellular second messenger of the target, detecting catalytic/enzymatic activity of the target an appropriate substrate (e.g., wee1 degradation, cdk1 phosphorylation) detecting the induction of a reporter gene (comprising a target-responsive regulatory element operatively linked to a nucleic acid encoding a detectable marker, e.g., luciferase), or detecting a target-regulated cellular response (e.g., mitosis).

In yet another embodiment, an assay of the present invention is a cell-free assay in which Tome-1 or biologically active portion thereof is contacted with a test compound and the ability of the test compound to bind to Tome-1 or a biologically active portion of Tome-1 is determined. In one embodiment biologically active portions of Tome-1 to be used in assays of the present invention include amino acid residues in one or more destruction boxes; the F box (e.g., residues in the 90-113 region of SEQ ID NO: 3); and the KEN motif (e.g., amino acid residues 372-374 of SEQ ID NO:3). Binding of the test compound to Tome-1 can be determined either directly or indirectly as described above. In one embodiment, the assay includes contacting Tome-1 or biologically active portion of Tome-1 with a known compound which binds Tome-1 to form an assay mixture, contacting the assay mixture with a test compound, and determining the ability of the test compound to interact with Tome-1, wherein determining the ability of the test compound to interact with Tome-1 comprises determining the ability of the test compound to preferentially bind to Tome-1 or a biologically active portion of Tome-1 as compared to the known compound.

In another embodiment, the assay is a cell-free assay in which Tome-1 or a biologically active portion of Tome-1 is contacted with a test compound and the ability of the test compound to modulate (e.g., stimulate or inhibit) the activity of Tome-1 or a biologically active portion of Tome-1 is determined. Determining the ability of the test compound to modulate the activity of Tome-1 can be accomplished, for example, by determining the ability of Tome-1 to bind to a Tome-1 target molecule by one of the methods described above for determining direct binding. Determining the ability of Tome-1 to bind to a Tome-1 target molecule can also be accomplished using a technology such as real-time Biomolecular Interaction Analysis (BIA) (Sjolander, S. and Urbaniczky, C. (1991) Anal. Chem. 63:2338-2345 and Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of Tome-1 can be accomplished by determining the ability of Tome-1 to further modulate the activity of a downstream effector of a Tome-1 target molecule. For example, the activity of the effector molecule on an appropriate target can be determined or the binding of the effector to an appropriate target can be determined as previously described.

In yet another embodiment, the cell-free assay involves contacting Tome-1 or a biologically active portion of Tome-1 with a known compound which binds Tome-1 to form an assay mixture, contacting the assay mixture with a test compound, and determining the ability of the test compound to interact with Tome-1, wherein determining the ability of the test compound to interact with Tome-1 comprises determining the ability of Tome-1 to preferentially bind to or modulate the activity of a Tome-1 target molecule (e.g., wee1 ubiquitinylation, wee1 degradation, a decrease in cdk1 phosphorylation and the like).

In more than one embodiment of the above assay methods of the present invention, it may be desirable to immobilize either Tome-1 or its target molecule to facilitate separation of complexed from uncomplexed forms of one or both of the proteins, as well as to accommodate automation of the assay. Binding of a test compound to Tome-1, or interaction of Tome-1 with a target molecule in the presence and absence of a candidate compound, can be accomplished in any vessel suitable for containing the reactants. Examples of such vessels include microtitre plates, test tubes, and microfuge tubes. In one embodiment, a fusion protein can be provided which adds a domain that allows one or both of the proteins to be bound to a matrix. For example, glutathione-S-transferase/Tome-1 fusion proteins or glutathione-S-transferase/target fusion proteins can be adsorbed onto glutathione sepharose beads (Sigma, St. Louis, Mo.) or glutathione-derivatized microtitre plates, which are then combined with the test compound or the test compound and either the non-adsorbed target protein or Tome-1, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of Tome-1 binding or activity determined using standard techniques.

Other techniques for immobilizing proteins on matrices can also be used in the screening assays of the invention. For example, either Tome-1 or a Tome-1 target molecule can be immobilized utilizing conjugation of biotin and avidin or streptavidin. Biotinylated Tome-1 or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce). Alternatively, antibodies reactive with Tome-1 or target molecules that do not interfere with binding of Tome-1 to its target molecule can be derivatized to the wells of the plate, and unbound target or Tome-1 trapped in the wells by antibody conjugation. Methods for detecting such complexes, in addition to those described above for the GST-immobilized complexes, include immunodetection of complexes using antibodies reactive with Tome-1 or Tome-1 target molecule, as well as enzyme-linked assays which rely on detecting an enzymatic activity associated with Tome-1 or Tome-1 target molecule.

In another embodiment, modulators of Tome-1 expression and/or Tome-1 degradation are identified in a method wherein a cell is contacted with a candidate compound and the expression of Tome-1 and/or Tome-1 mRNA in the cell is determined. The level of Tome-1 protein and/or Tome-1 mRNA in the presence of the candidate compound is compared to the level of Tome-1 protein and/or Tome-1 mRNA in the absence of the candidate compound. The candidate compound can then be identified as a modulator of Tome-1 protein expression, Tome-1 mRNA expression, and/or Tome-1 degradation based on this comparison. For example, when expression of Tome-1 protein and/or Tome-1 mRNA is greater and/or the rate of Tome-1 degradation is lower (statistically significantly greater or lower, respectively) in the presence of the candidate compound than in its absence, the candidate compound is identified as a stimulator of Tome-1 protein expression and/or Tome-1 mRNA expression and/or an inhibitor of Tome-1 degradation, respectively. Alternatively, when expression of Tome-1 protein and/or Tome-1 mRNA is less and/or the rate of Tome-1 degradation is higher (statistically significantly lower or greater, respectively) in the presence of the candidate compound than in its absence, the candidate compound is identified as an inhibitor of Tome-1 protein expression and/or Tome-1 mRNA expression and/or a stimulator of Tome-1 degradation, respectively. The level of Tome-1 mRNA or protein expression and Tome-1 degradation in the cells can be determined by methods described herein for detecting Tome-1 mRNA or protein.

In yet another aspect of the invention, Tome-1 can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO 94/10300), to identify other proteins, which bind to or interact with Tome-1 (“Tome-1-binding proteins” or “Tome-1-bp”) and are involved in Tome-1 activity (e.g., wee1 ubiquitinylation, wee1 degradation and/or mitotic entry). Such Tome-1-binding proteins are also likely to be involved in the propagation of signals by Tome-1 or Tome-1 targets as, for example, downstream elements of a Tome-1 -mediated signaling pathway. Alternatively, such Tome-1-binding proteins are likely to be Tome-1 inhibitors (such members of the anaphase promoting complex (APC)).

The two-hybrid system is based on the modular nature of most transcription factors, which consist of separable DNA-binding and activation domains. Briefly, the assay utilizes two different DNA constructs. In one construct, the gene that codes for Tome-1 is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a Tome-1-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with Tome-1.

In another embodiment, an assay is an animal model based assay comprising contacting a an animal with a test compound and determining the ability of the test compound to alter Tome-1 expression and/or Tome-1 degradation. In one embodiment animals include but are not limited to mammals such as non-human primates, rabbits, rats, mice, and the like and amphibians such as Xenopus.

This invention further pertains to novel agents identified by the above-described screening assays. Accordingly, it is within the scope of this invention to further use an agent identified as described herein in an appropriate animal model as described herein. For example, an agent identified as described herein (e.g., a Tome-1 modulating agent) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments of disorders associated with aberrant cellular proliferation as described herein.

Recombinant Expression Vectors and Host Cells

Another aspect of the invention pertains to vectors, such as expression vectors, containing a nucleic acid encoding a Tome-1 protein (or a portion thereof). As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., Tome-1 proteins, mutant forms of Tome-1 proteins, fusion proteins, and the like).

The recombinant expression vectors of the invention can be designed for expression of Tome-1 in prokaryotic or eukaryotic cells. For example, Tome-1 or Tome-1 fragments can be expressed in bacterial cells such as E. coli, insect cells (using baculovirus expression vectors), yeast cells, amphibian cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of polypeptides in prokaryotes is most often carried out in E. coli with vectors containing constitutive or inducible promoters directing the expression of either fusion or non-fusion proteins. Fusion vectors add a number of amino acids to a polypeptide encoded therein, usually to the amino terminus of the recombinant polypeptide. Such fusion vectors typically serve three purposes: 1) to increase expression of recombinant polypeptide; 2) to increase the solubility of the recombinant polypeptide; and 3) to aid in the purification of the recombinant polypeptide by acting as a ligand in affinity purification. Often, in fusion expression vectors, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Purified fusion polypeptide can be utilized in translation initiation activity assays, or to generate antibodies specific for Tome-1, for example.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111). Such alteration of nucleic acid sequences of the invention can be carried out by standard DNA synthesis techniques.

In another embodiment, the Tome-1 expression vector is a yeast expression vector. Examples of vectors for expression in yeast S. cerevisiae include pYepSec1 (Baldari et al., (1987) Embo J. 6:229), pMFa (Kurjan and Herskowitz, (1982) Cell 30:933), pJRY88 (Schultz et al., (1987) Gene 54:113), pYES2 (Invitrogen Corporation, San Diego, Calif.), and picZ (InVitrogen Corp, San Diego, Calif.).

Alternatively, Tome-1 polypeptides can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156) and the pVL series (Lucklow and Summers (1989) Virology 170:31).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187). When used in mammalian cells, the expression vector's control functions are often provided by viral regulatory elements. For example, commonly used promoters are derived from polyoma, adenovirus 2, cytomegalovirus and Simian virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268), lymphoid-specific promoters (Calame and Eaton (1988) Adv. Immunol. 43:235), in particular promoters of T cell receptors (Winoto and Baltimore (1989) EMBO J. 8:729) and immunoglobulins (Banerji et al. (1983) Cell 33:729; Queen and Baltimore (1983) Cell 33:741), neuron-specific promoters (e.g., the neurofilament promoter; Byrne and Ruddle (1989) Proc. Natl. Acad. Sci. U.S.A. 86:5473), pancreas-specific promoters (Edlund et al. (1985) Science 230:912), and mammary gland-specific promoters (e.g., milk whey promoter; U.S. Pat. No. 4,873,316 and European Application Publication No. 264,166). Developmentally regulated promoters are also encompassed, for example the murine hox promoters (Kessel and Gruss (1990) Science 249:374) and the α-fetoprotein promoter (Campes and Tilghman (1989) Genes Dev. 3:537).

In one embodiment, the present invention provides a nucleic acid molecule which is antisense to a Tome-1 nucleic acid molecule. As used herein, the term “antisense” refers to a nucleic acid that interferes with the function of DNA and/or RNA and may result in suppression of expression of the RNA and/or DNA. The antisense nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire Tome-1 coding strand, or to only a portion thereof.

An antisense nucleic acid molecule can be delivered to a cell to express an exogenous nucleotide sequence, to inhibit, eliminate, augment, or alter expression of an endogenous nucleotide sequence, or to express a specific physiological characteristic not naturally associated with the cell. In one embodiment, the antisense nucleic acid is an antisense RNA, an interfering double stranded RNA (“dsRNA”) or a short interfering RNA (“siRNA”).

As used herein, the term “siRNA” refers to double-stranded RNA that is less than 30 bases and preferably 21-25 bases in length. siRNA may be prepared by any method known in the art. For a review, see Nishikura (2001) Cell 16:415. In one embodiment, single-stranded, gene-specific sense and antisense RNA oligomers with overhanging 3′ deoxynucleotides are prepared and purified. For example, two oligomers, can be annealed by heating to 94° C. for 2 minutes, cooling to 90° C. for 1 minute, and then cooling to 20° C. at a rate of 1° C. per minute. The siRNA can then be injected into an animal or delivered into a desired cell type using methods of nucleic acid delivery described herein.

Another aspect of the invention pertains to host cells into which a recombinant expression vector of the invention has been introduced, containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, host cells can be bacterial cells such as E. coli, insect cells, yeast, Xenopus cells, or mammalian cells (such as Chinese hamster ovary cells (CHO), African green monkey kidney cells (COS), or fetal human cells (293T)). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a detectable translation product or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a Tome-1 protein. Accordingly, the invention further provides methods for producing a Tome-1 protein using the host cells of the invention. In one embodiment, the method comprises culturing the host cell of invention (into which a recombinant expression vector encoding a detectable translation product has been introduced) in a suitable medium such that a detectable translation product is produced. In another embodiment, the method further comprises isolating a Tome-1 protein from the medium or the host cell.

The host cells of the invention can also be used to produce nonhuman transgenic animals. For example, in one embodiment, a host cell of the invention is a fertilized oocyte or an embryonic stem cell into which Tome-1-coding sequences have been introduced. Such host cells can then be used to create non-human transgenic animals in which exogenous Tome-1 sequences have been introduced into their genome. Such animals are useful for studying the function and/or activity of Tome-1 and for identifying and/or evaluating modulators of Tome-1 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a Tome-1-encoding nucleic acid into the male pronuclei of a fertilized oocyte, e.g., by microinjection, retroviral infection, and allowing the oocyte to develop in a pseudopregnant female foster animal. The Tome-1 cDNA sequence of SEQ ID NOs:4, 5 or 6 can be introduced as a transgene into the genome of a non-human animal. Alternatively, a nonhuman homologue of a human Tome-1 gene, such as a mouse, rat or Xenopus Tome-1 gene, can be used as a transgene. Alternatively, a Tome-1 gene homologue, such as another Tome-1 family member, can be isolated based on hybridization to the Tome-1 cDNA sequences of SEQ ID NOs:4, 5 or 6 and used as a transgene. Intronic sequences and polyadenylation signals can also be included in the transgene to increase the efficiency of expression of the transgene. A tissue-specific regulatory sequence(s) can be operably linked to a detectable translation product transgene to direct expression of a detectable translation product to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al., and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a detectable translation product transgene in its genome and/or expression of detectable translation product mRNA in tissues or cells of the animals. A transgenic founder animal can then be used to breed additional animals carrying the transgene. Moreover, transgenic animals carrying a transgene encoding a detectable translation product can further be bred to other transgenic animals carrying other transgenes.

To create a homologous recombinant animal, a vector is prepared which contains at least a portion of a Tome-1 gene into which a deletion, addition or substitution has been introduced to thereby alter, e.g., functionally disrupt, the Tome-1 gene. The Tome-1 gene can be a human gene (e.g., the cDNA of SEQ ID NO:5), or a non-human homologue of a human Tome-1 gene (e.g., the cDNA of SEQ ID NOs:4 or 6). For example, a mouse Tome-1 gene (SEQ ID NO:4) can be used to construct a homologous recombination vector suitable for altering an endogenous Tome-1 gene in the mouse genome. In one embodiment, the vector is designed such that, upon homologous recombination, the endogenous Tome-1 gene is functionally disrupted (i.e., no longer encodes a functional protein; also referred to as a “knock out” vector). Alternatively, the vector can be designed such that, upon homologous recombination, the endogenous Tome-1 gene is mutated or otherwise altered but still encodes functional protein (e.g., the upstream regulatory region can be altered to thereby alter the expression of the endogenous Tome-1 protein). In the homologous recombination vector, the altered portion of the Tome-1 gene is flanked at its 5′ and 3′ ends by additional nucleic acid sequence of the Tome-1 gene to allow for homologous recombination to occur between the exogenous Tome-1 gene carried by the vector and an endogenous Tome-1 gene in an embryonic stem cell. The additional flanking Tome-1 nucleic acid sequence is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced Tome-1 gene has homologously recombined with the endogenous Tome-1 gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823 and in PCT International Publication Nos.: WO 90/11354 by Le Mouellec et al.; WO 91/01140 by Smithies et al.; WO 92/0968 by Zijlstra et al.; and WO 93/04169 by Bems et al.

In another embodiment, transgenic non-humans animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. U.S.A. 89:6232. Another example of a recombinase system is the FLP recombinase system of S. cerevisiae (O'Gorman et al. (1991) Science 251:1351). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810. In brief, a cell, e.g., a somatic cell, from the transgenic animal can be isolated and induced to exit the growth cycle and enter G₀ phase. Alternatively, a cell, e.g., an embryonic stem cell, from the inner cell mass of a developing embryo can be transformed with a preferred transgene. Alternatively, a cell, e.g., a somatic cell, from cell culture line can be transformed with a preferred transgene and induced to exit the growth cycle and enter G₀ phase. The cell can then be fused, e.g., through the use of electrical pulses, to an enucleated mammalian oocyte. The reconstructed oocyte is then cultured such that it develops to morula or blastocyst and then transferred to pseudopregnant female foster animal. The offspring borne of this female foster animal will be a clone of the animal from which the nuclear donor cell, e.g., the somatic cell, is isolated.

Diagnostic Assays

An exemplary method for detecting the presence or absence of Tome-1 protein, Tome-1 nucleic acid, or Tome-1 degradation in a biological sample involves obtaining a biological sample from a test subject and contacting the biological sample with a compound or an agent capable of detecting Tome-1 protein or nucleic acid (e.g., mRNA, genomic DNA) that encodes Tome-1 protein such that the presence of Tome-1 protein or nucleic acid is detected in the biological sample. In one embodiment, an agent for detecting Tome-1 mRNA or genomic DNA is a labeled nucleic acid probe capable of hybridizing to Tome-1 mRNA or genomic DNA. The nucleic acid probe can be, for example, a full-length Tome-1 nucleic acid, such as the nucleic acid of SEQ ID NOs:4, 5 or 6, or a portion thereof, such as an oligonucleotide of at least 15, 30, 50, 100, 250, 300, 350, 400, 450, 500, 550, 600, 650, 700, 750, 760, 770, 780, 790, 800, 810, 820, 830, 840, 850, 900, 950, 960, 970, 980, 990, 1000, 1010, 1020, 1030, 1040, 1050, 1100, 1150, 1200, 1250, 1300, 1350, 1400, 1410, 1420, 1430, 1440, 1450 or more nucleotides in length and sufficient to specifically hybridize under stringent conditions to Tome-1 mRNA or genomic DNA. Other suitable probes for use in the diagnostic assays of the invention are described herein.

A desired agent for detecting Tome-1 protein is an antibody capable of binding to Tome-1 protein, such as an antibody with a detectable label. Antibodies can be polyclonal or monoclonal. An intact antibody, or a fragment thereof (e.g., Fab or F(ab′)₂) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect Tome-1 mRNA, protein, or genomic DNA in a biological sample in vitro as well as in vivo. For example, in vitro techniques for detection of Tome-1 mRNA include Northern hybridizations and in situ hybridizations. In vitro techniques for detection of Tome-1 protein include enzyme linked immunosorbent assays (ELISAs), Western blots, immunoprecipitations and immunofluorescence. In vitro techniques for detection of Tome-1 genomic DNA include Southern hybridizations. Furthermore, in vivo techniques for detection of Tome-1 protein include introducing into a subject a labeled anti-Tome-1 antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. In one embodiment biological sample is a serum sample or a biopsy sample isolated by conventional means from a subject.

In another embodiment, the methods further involve obtaining a control biological sample from a control subject, contacting the control sample with a compound or agent capable of detecting Tome-1 protein, mRNA, or genomic DNA, such that the presence of Tome-1 protein, mRNA or genomic DNA is detected in the biological sample, and comparing the presence of Tome-1 protein, mRNA or genomic DNA in the control sample with the presence of Tome-1 protein, mRNA or genomic DNA in the test sample.

The invention also encompasses kits for detecting the presence of Tome-1 in a biological sample. For example, the kit can comprise a labeled compound or agent capable of detecting Tome-1 protein or mRNA in a biological sample; means for determining the amount of Tome-1 in the sample; and means for comparing the amount of Tome-1 in the sample with a standard. The compound or agent can be packaged in a suitable container. The kit can further comprise instructions for using the kit to detect Tome-1 protein or nucleic acid.

Prognostic Assays

The diagnostic methods described herein can furthermore be utilized to identify subjects having or at risk of developing a disease or disorder associated with aberrant Tome-1 expression or activity (e.g., aberrant cellular proliferation). For example, the assays described herein, such as the preceding diagnostic assays or the following assays, can be utilized to identify a subject having or at risk of developing a disorder associated with a misregulation in Tome-1 protein activity or nucleic acid expression, such as disorders associated with aberrant proliferation such as cancer.

Thus, the present invention provides a method for identifying a disease or disorder associated with aberrant Tome-1 expression or activity in which a test sample is obtained from a subject and Tome-1 protein or nucleic acid (e.g., mRNA or genomic DNA) is detected, wherein the presence or increased levels of Tome-1 protein or nucleic acid is diagnostic for a subject having or at risk of developing a disease or disorder associated with aberrant Tome-1 expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant Tome-1 expression or activity. For example, such methods can be used to determine whether a subject can be effectively treated with an agent for cancer. Thus, the present invention provides methods for determining whether a subject can be effectively treated with an agent for a disorder associated with aberrant Tome-1 expression or activity in which a test sample is obtained and Tome-1 protein or nucleic acid expression or activity is detected (e.g., wherein the abundance of Tome-1 protein or nucleic acid expression or activity is diagnostic for a subject that can be administered the agent to treat a disorder associated with aberrant Tome-1 expression or activity).

The methods of the invention can also be used to detect genetic alterations in a Tome-1 gene, thereby determining if a subject with the altered gene is at risk for a disorder characterized by misregulation in Tome-1 protein activity or nucleic acid expression, such as cancer. In certain embodiments, the methods include detecting, in a sample of cells from the subject, the presence or absence of a genetic alteration characterized by an alteration affecting the integrity of a gene encoding a Tome-1 protein and/or the misexpression of the Tome-1 gene. For example, such genetic alterations can be detected by ascertaining the existence of at least one of: 1) a deletion of one or more nucleotides from a Tome-1 gene; 2) an addition of one or more nucleotides to a Tome-1 gene; 3) a substitution of one or more nucleotides of a Tome-1 gene, 4) a chromosomal rearrangement of a Tome-1 gene; 5) an alteration in the level of a messenger RNA transcript of a Tome-1 gene; 6) aberrant modification of a Tome-1 gene, such as of the methylation pattern of the genomic DNA; 7) the presence of a non-wild type splicing pattern of a messenger RNA transcript of a Tome-1 gene; 8) a non-wild type level of a Tome-1 protein; 9) allelic loss of a Tome-1 gene; and 10) inappropriate post-translational modification of a Tome-1 protein. As described herein, there are a large number of assays known in the art which can be used for detecting alterations in a Tome-1 gene.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077; and Nakazawa et al. (1994) Proc. Natl. Acad. Sci. USA 91:360), the latter of which can be particularly useful for detecting point mutations in the Tome-1 gene (see Abravaya et al. (1995) Nucleic Acids Res. 23:675). This method can include the steps of collecting a sample of cells from a subject, isolating nucleic acid (e.g., genomic, mRNA or both) from the cells of the sample, contacting the nucleic acid sample with one or more primers which specifically hybridize to a Tome-1 gene under conditions such that hybridization and amplification of the Tome-1 gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci. USA 87:1874), transcriptional amplification system (Kwoh, D. Y. et al., (1989) Proc. Natl. Acad. Sci. USA 86:1173), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a Tome-1 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in Tome-1 can be identified by hybridizing a sample and control nucleic acids, e.g., DNA or RNA, to high density arrays containing hundreds or thousands of oligonucleotides probes (Cronin, M. T. et al. (1996) Human Mutation 7: 244; Kozal, M. J. et al. (1996) Nature Medicine 2:753). For example, genetic mutations in Tome-1 can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the Tome-1 gene and detect mutations by comparing the sequence of the sample Tome-1 with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147).

Other methods for detecting mutations in the Tome-1 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type Tome-1 sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397; Saleeba et al. (1992) Methods Enzymol. 217:286. In one embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in Tome-1 cDNAs obtained from samples of cells. For example, the mutY enzyme of E. coli cleaves A at G/A mismatches and the thyrnidine DNA glycosylase from HeLa cells cleaves T at G/T mismatches (Hsu et al. (1994) Carcinogenesis 15:1657). According to an exemplary embodiment, a probe based on a Tome-1 sequence, e.g., a wild-type Tome-1 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in Tome-1 genes. For example, single strand conformation polymorphism (SSCP) may be used to detect differences in electrophoretic mobility between mutant and wild type nucleic acids (Orita et al. (1989) Proc. Natl. Acad. Sci. USA 86:2766, see also Cotton (1993) Mutat. Res. 285:125; and Hayashi (1992) Genet. Anal. Tech. Appl. 9:73). Single-stranded DNA fragments of sample and control Tome-1 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nuc. Acids Res. 17:2437) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a Tome-1 gene and/or aberrant sister cellular proliferation.

Furthermore, any cell type or tissue in which Tome-1 is expressed may be utilized in the prognostic assays described herein.

Therapeutic Methods

Another aspect of the invention pertains to methods of modulating mitosis for therapeutic purposes. Accordingly, in an exemplary embodiment, the modulatory method of the invention involves contacting a cell with an agent that inhibits Tome-1. An agent that inhibits Tome-1 can be an agent as described herein (e.g., an anaphase promoting complex (APC) agent), a naturally-occurring target molecule of Tome-1 (e.g., wee1 , Skp-1, Cul-1 and the like), a Tome-1 antibody, or other small molecule. In one embodiment, the agent modulates one or more agents (e.g., polypeptides and/or proteins) which controls mitotic entry. In another embodiment, the agent inhibits one or more Tome-1 activities. In another embodiment, the agent mediates Tome-1 proteolysis. In another embodiment, the agent inhibits Tome-1 binding to Skp-1 and/or Cul-1. In another embodiment, the agent dephosphorylates wee1 (e.g., serine residue 28 of Xenopus wee1). In another embodiment, the agent activates APC. These modulatory methods can be performed in vitro (e.g., by culturing a cell with the agent) or, alternatively, in vivo (e.g., by administering the agent to a subject). As such, the present invention provides methods of treating an individual afflicted with a disease or disorder characterized by aberrant cellular proliferation. Examples of such disorders are described herein. In one embodiment, the method involves administering an agent (e.g., an agent identified by a screening assay described herein), or combination of agents that inhibits translation initiation.

In one embodiment, the present invention involves a method for treatment of a cellular proliferation-associated disease or disorder which includes the step of administering a therapeutically effective amount of an agent which inhibits cellular proliferation (e.g., inhibits mitosis) to a subject. As defined herein, a therapeutically effective amount of agent (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an inhibitor can include a single treatment or, preferably, can include a series of treatments. It will also be appreciated that the effective dosage of in used for treatment may increase or decrease over the course of a particular treatment. Changes in dosage may result from the results of diagnostic assays as described herein.

Monitoring of Effects During Clinical Trials

Monitoring the influence of agents (e.g., drugs) on the expression or activity of Tome-1 (e.g., the modulation of mitosis and/or aberrant cellular proliferation) can be applied not only in basic drug screening, but also in clinical trials. For example, the effectiveness of an agent determined by a screening assay as described herein to decrease Tome-1 gene expression, or protein levels or increase wee1 activity, can be monitored in clinical trials of subjects exhibiting increased Tome-1 gene expression, protein levels, or decreased wee1 activity. Alternatively, the effectiveness of an agent determined by a screening assay to increase Tome-1 gene expression, or protein levels or decrease wee1 activity, can be monitored in clinical trials of subjects exhibiting decreased Tome-1 gene expression, protein levels, or increased wee1 activity.

In one embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of a Tome-1 protein, mRNA or genomic DNA in the preadministration sample; (iii) obtaining one or more post-administration samples from the subject; (iv) detecting the level of expression or activity of the Tome-1 protein, mRNA or genomic DNA in the post-administration samples; (v) comparing the level of expression or activity of the Tome-1 protein, mRNA, or genomic DNA in the pre-administration sample with the Tome-1 protein, mRNA, or genomic DNA in the post administration sample or samples; and (vi) altering the administration of the agent to the subject accordingly. For example, increased administration of the agent may be desirable to decrease the expression or activity of Tome-1 to higher lower than detected, i.e., to increase the effectiveness of the agent. Alternatively, decreased administration of the agent may be desirable to increase expression or activity of Tome-1 to higher levels than detected, i.e. to decrease the effectiveness of the agent. According to such an embodiment, Tome-1 expression or activity may be used as an indicator of the effectiveness of an agent, even in the absence of an observable phenotypic response.

Pharmaceutical Compositions

Methods of administering a compound to an individual include providing pharmaceutically acceptable compositions. In one embodiment, pharmaceutically acceptable compositions comprise a therapeutically effective amount of one or more of the compounds described above, formulated together with one or more pharmaceutically acceptable carriers (additives) and/or diluents. The pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam. In one embodiment, the therapeutic compound is administered orally. The compounds of the invention can be formulated as pharmaceutical compositions for administration to a subject, e.g., a mammal, including a human.

The compounds of the invention are administered to subjects in a biologically compatible form suitable for pharmaceutical administration in vivo. By “biologically compatible form suitable for administration in vivo” is meant a compound to be administered in which any toxic effects are outweighed by the therapeutic effects of the compound. The term “subject” is intended to include living organisms such as mammals. Examples of subjects include humans, monkeys, pigs, dogs, cats, rabbits, mice, rats, frogs, toads and transgenic species thereof. Administration of a therapeutically active amount of the therapeutic compositions of the present invention is defined as an amount effective, at dosages and for periods of time necessary to achieve the desired result. For example, a therapeutically active amount of a compound of the invention may vary according to factors such as the disease state, age, sex, and weight of the individual, and the ability of antibody to elicit a desired response in the individual. Dosage regimes may be adjusted to provide the optimum therapeutic response. For example, several divided doses may be administered daily or the dose may be proportionally reduced as indicated by the exigencies of the therapeutic situation.

The active compound may be administered in a convenient manner such as by injection (subcutaneous, intravenous, etc.), oral administration, inhalation, transdermal application, or rectal administration. Depending on the route of administration, the active compound may be coated in a material to protect the compound from the action of enzymes, acids and other natural conditions which may inactivate the compound.

A compound of the invention can be administered to a subject in an appropriate carrier or diluent, co-administered with enzyme inhibitors or in an appropriate carrier such as liposomes. The term “pharmaceutically acceptable carrier” as used herein is intended to include diluents such as saline and aqueous buffer solutions. To administer a compound of the invention by other than parenteral administration, it may be necessary to coat the compound with, or co-administer the compound with a material to prevent its inactivation. Liposomes include water-in-oil-in-water emulsions as well as conventional liposomes (Strejan et al. (1984) J. Neiuroimmunol. 7:27). The active compound may also be administered parenterally or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The pharmaceutically acceptable carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (e.g., antibody) plus any additional desired ingredient from a previously sterile-filtered solution thereof.

When the active compound is suitably protected, as described above, the composition may be orally administered, for example, with an inert diluent or an assimilable edible carrier. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the therapeutic treatment of individuals.

The following examples are provided for exemplification purposes only and are not intended to limit the scope of the invention which has been described in broad terms above.

### EXAMPLE I

### Tome-1 is a Novel Substrate of APC^{CDH1}

To identify novel substrates of the G1 active APC, an in vitro expression cloning strategy used previously to clone three novel mitotic substrates of the APC: geminin, vertebrate securin, and Xkid was modified (Funabiki et al. (2000) Cell 102:411; McGarry et al. (1998) Cell 93:1043; Zou et al. (1999) Science 285:418). After in vitro translating pools of Xenopus laevis cDNAs in an ³⁵S-labeling reticulocyte lysate translation system, the translated products were incubated with Xenopus egg extracts in the presence or absence of recombinant CDH1. Since there is little, if any, expression of CDH1 in the egg, Xenopus egg extracts were used as a source of CDH1-deficient APC (Lorca et al. (1998) EMBO J. 17:3565). After termination of the degradation reactions, SDS-PAGE analysis, and autoradiography, proteins were identified that were degraded specifically in the presence of APC^{CDH1}. The cDNAs corresponding to these proteins were isolated through a selection procedure described in Lustig et al. ((1997) Methods Enzymol. 283:83). One of the proteins was named Tome-1, because it functions as a trigger of mitotic entry.

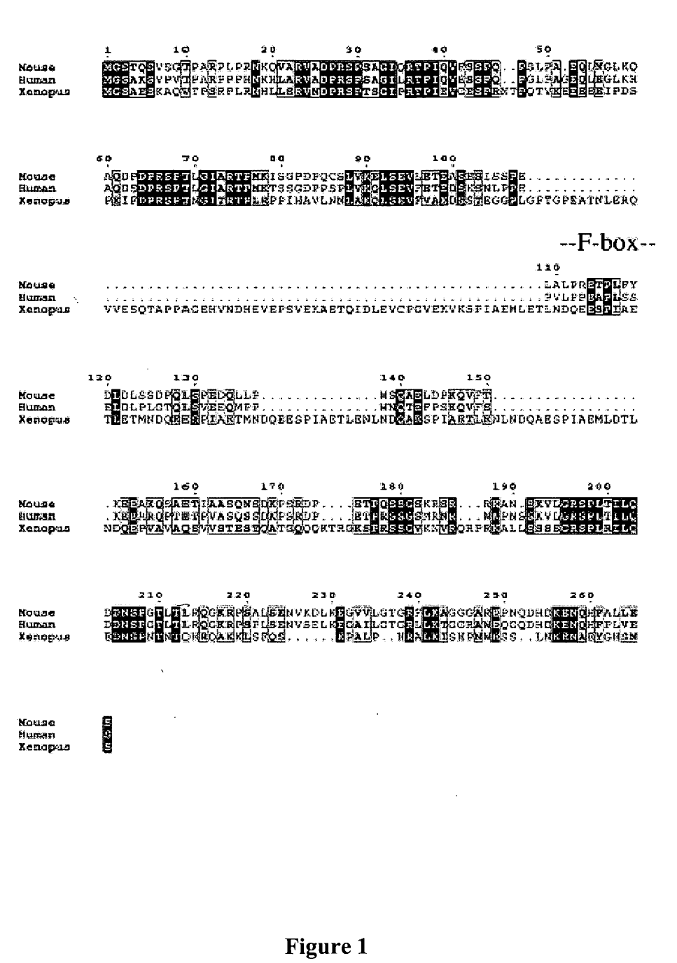

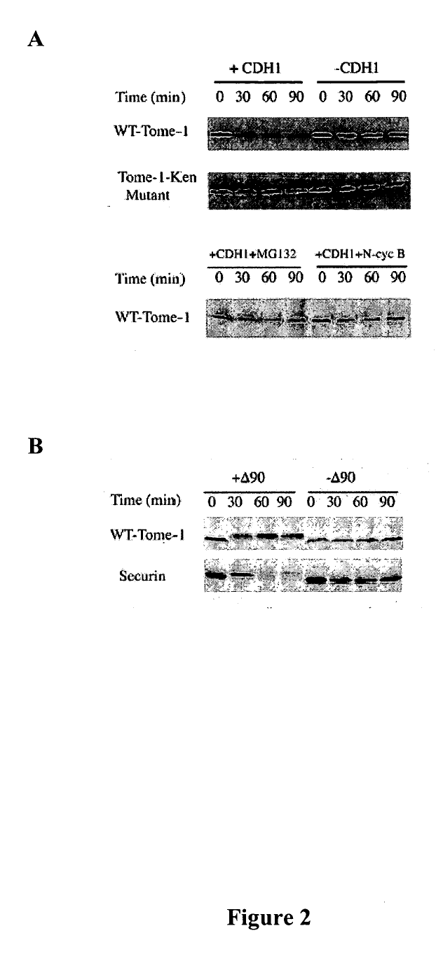

Xenopus Tome-1 was determined to be 30% identical to human Tome-1, containing two N-terminal destruction boxes, and a C-terminal KEN (FIG. 1). The presence of a KEN sequence in Tome-1 indicated that Tome-1 was a bona fide substrate of the APC. To test this claim, it was determined whether Tome-1 degradation could be competitively inhibited by adding increasing concentrations of a known APC substrate. As illustrated in FIG. 2A, Tome-1 was degraded in a CDH1 -dependent manner, but this degradation was inhibited greatly in the presence of the N-terminal region of cyclin B. To test whether degradation of Tome-1 was proteasome mediated, degradation reactions were performed in the presence of 100 μM of the proteasomal inhibitor MG132 (Lee et al. (1998) Trends Cell Biol. 8:397). Under these conditions, which are known to inhibit proteasomal activity, no degradation of Tome-1 was observed, indicating that active proteasomes are required for degradation of Tome-1.

To determine if Tome-1 is strictly an APC^{CDH1} substrate or both an APC^{CDH1} and an APC^{CCD20} substrate, degradation of Tome-1 was assayed in mitotic extracts. A non-degradable version of cyclin B (Δ90) was used to drive interphase extracts into mitosis, thereby activating APC^{CDC20}. Degradation of Tome-1 was then assayed in these extracts. As illustrated in FIG. 2B, although Tome-1 was phosphorylated as indicated by its reduced electrophoretic mobility on SDS-PAGE, it was not degraded in mitotic extracts, indicating that it is only recognized by APC^{CDH1} and not by APC^{CDC20}.

Recognition of substrates by APC^{CDH1} requires the presence of a KEN or RXXL motif in most substrates studied thus far (Pfleger et al. (2000) Genes Dev. 14:655; Pfleger et al. (2001) Genes Dev. 15:2396). To determine if the KEN sequence present in Tome-1 is required for its degradation, each of these three residues was mutated to alanine and it was determined whether the mutated Tome-1 could be degraded in CDH1-supplemented extracts. FIG. 2A demonstrates that mutation of the KEN sequence to alanine stabilized Tome-1 in CDH1 supplemented extracts, indicating that the C-terminal KEN sequence present in Tome-1 is necessary for recognition by CDH1. By contrast, mutation of the destruction boxes had no effect on CDH1-dependent degradation in Xenopus egg extracts.

### EXAMPLE II

### Tome-1 is a Cytosolic Protein Degraded During G1

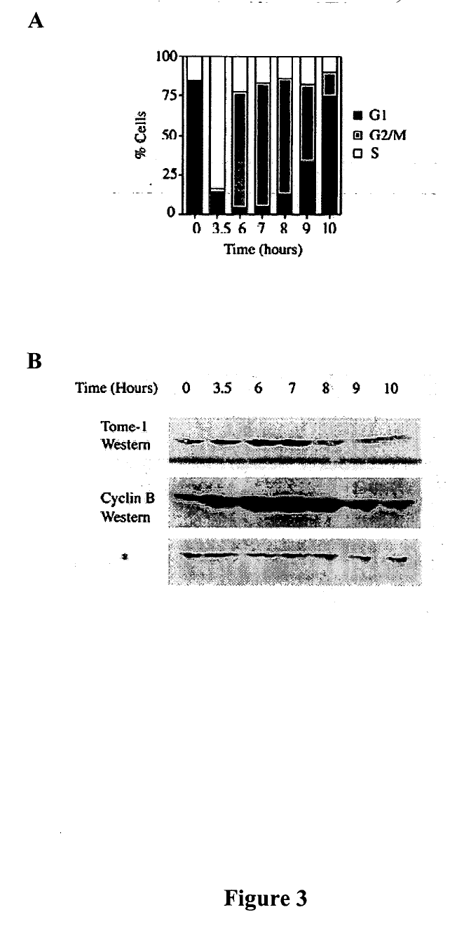

The degradation assays indicated that Tome-1 is a substrate of APC^{CDH1} in vitro. To determine if Tome-1 is also degraded in vivo, Tome-1 protein levels were assayed throughout the somatic cell cycle. HeLa cells were arrested at the G1/S transition with a double thymidine block, followed by a release wherein the thymidine was washed away. The cell cycle profile was then determined by FACS analysis. The relative amounts of cyclin B and Tome-1 were also compared by Western analysis. As depicted in FIGS. 3A and 3B, similar to cyclin B levels, Tome-1 levels were highest 6 hours after cells were washed free of thymidine. This corresponds to the time when there is the greatest proportion of cells in the G2 or M phases of the cell cycle. By contrast, both cyclin B and Tome-1 levels decreased as the proportion of cells in the G1 phase of the cell cycle increased, indicating that both proteins were degraded at this time.

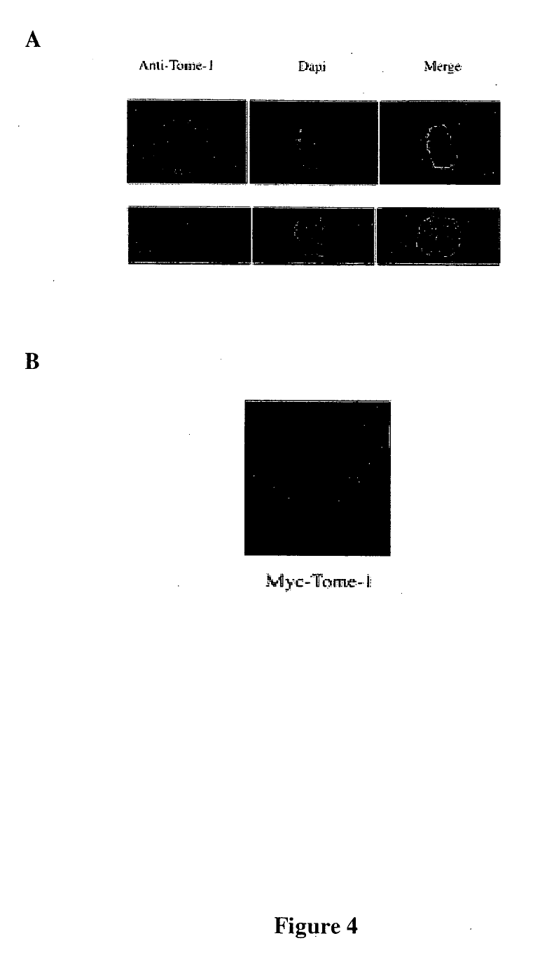

A second indication that Tome-1 levels fluctuate throughout the cell cycle came when CFPAC cells were immunostained with the anti-Tome-1 antibody. The intensity of the immunofluorescence signal for Tome-1 was greater in mitotic cells than in non-mitotic cells (FIG. 4A). It was also observed that in almost all cells, the endogenous Tome-1 was predominantly cytosolic (FIG. 4A). Further, when Tome-1 was overexpressed in tissue culture cells, it was exclusively cytosolic (FIG. 4B).

### EXAMPLE III

### Tome-1 Associates with Skp-1

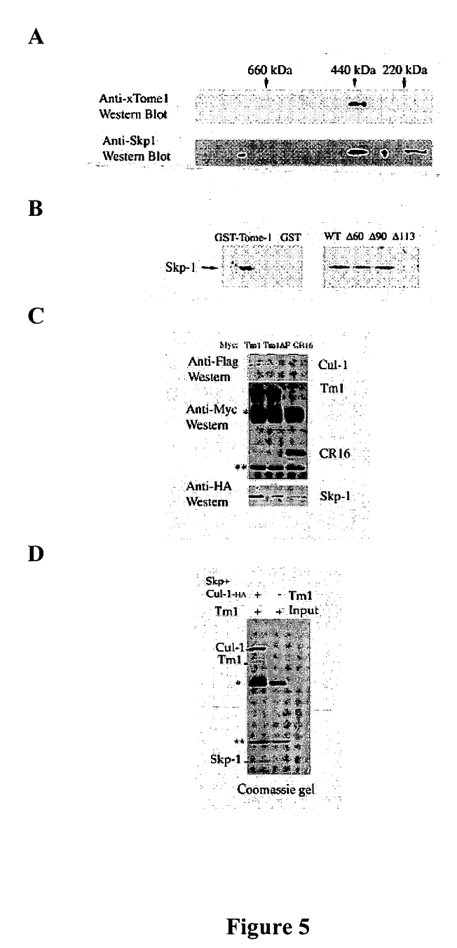

The observation that Tome-1 is a cytosolic protein whose expression is greatest at mitosis indicated that it may have an essential function at this time. Without intending to be bound by theory, it was reasoned that the mitotic cellular pathway in which Tome-1 functions could be discovered by identifying Tome1-interacting proteins. Therefore, Tome-1 was purified from nocodazole-arrested XTC cells and associated proteins were assayed for. When these extracts were fractionated by size exclusion chromatography, it was observed that Tome-1 eluted as part of a complex with an apparent molecular weight of 400 kDa. Tome-1 complexes were detected by immunoblot analysis of column fractions using an anti-Tome-1 antibody (FIG. 5A). Since a two hybrid screen indicated that Tome-1 could interact with the SCF components Skp-1 and Cul-1, the Tome-1 complex was assayed for the presence of these proteins. Using anti-Skp-1, anti-Cul-1, and antiTome-1 antibodies, it was determined that Skp-1 and Cul-1 did indeed co-fractionate with Tome-1 in the early steps of the purification. In fact, when the molecular weight of the Tome-1-containing complex was calculated by combining size exclusion and sucrose density gradient profiles (sedimentation coefficient of 5S), a mass of 140 kDa was found, which is the predicted mass of a Tome-1, Skp-1, Cul-1 complex.

To determine if Tome-1 interacted with Skp-1 in vitro, an in vitro binding assay was performed with purified Tome-1 and ³⁵S-labeled Skp-1. A GST-Tome-1 fusion protein was expressed in E. coli, immobilized on glutathione beads, and subsequently incubated with in vitro translated Skp-1. After washing away the unbound Skp-1, significant binding of Skp-1 was observed to beads containing GST-mTome-1 relative to beads containing only GST (FIG. 5B). Most proteins known to associate with Skp-1 are F box proteins, so Tome-1 was analyzed to determine whether it contained an F box. Without intending to be bound by theory, close inspection of Tome-1 sequences indicated that a putative F box motif existed in all Tome-1 proteins, signifying that Tome-1 could be a member of the F box family of proteins (FIG. 1). To determine if the F box motif was necessary for Tome-1 to interact with Skp-1, versions of Tome-1 lacking putative F boxes were engineered and tested for the ability to interact with Skp-1, as judged by a glutathione co-precipitation assay. As shown in FIG. 5B, when residues 1-113 were removed from mouse Tome-1, binding to Skp-1 was lost. In contrast, removing residues 1-90 from mouse Tome-1 did not affect the Skp-1-Tome-1 interaction, indicating that residues in the 90-113 region are necessary for binding Skp-1, and likely contain the F box.

It was confirmed that this region contained the F box, since a mutated form of Tome-1 lacking critical F box residues did not bind Skp-1. As shown in FIG. 5C, mutating two essential F box residues (LP) to alanine reduced the extent to which Tome-1 interacted with Skp-1. Also, less Cul-1 precipitated with the F box mutant of Tome-1 relative to wild-type Tome-a (FIG. 5C).

Although Xenopus Tome-1 does not contain the LP residues found in mouse and human Tome-1, it does associate with Skp-1 and Cul-1 in vitro. Recombinant Xenopus Tome-1 were incubated in extracts isolated from SF9 cells expressing an HA-tagged version of Cul-1 and untagged Skp-1. Subsequently, the amount of Xenopus Tome-1 associated with anti-hemagglutinin (HA) immunoprecipitates either containing or lacking HA-Cul-1 and Skp-1 were determined by Coomassie staining and liquid chromatography-mass spectrometry/mass spectrometry (LC-MSIMS). As depicted in FIG. 5D, Xenopus Tome-1 efficiently interacted with HA-Cul-1 and Skp 1, but not with HA beads alone.

### EXAMPLE IV

### ΔTome-1 Inhibits Mitotic Entry

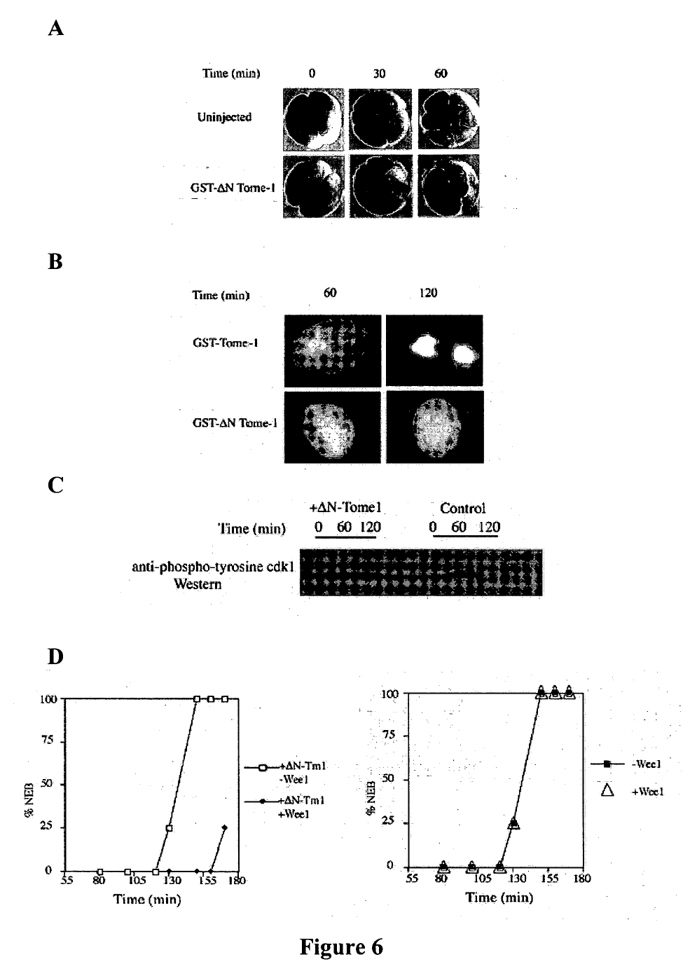

Without intending to be bound by theory, since the N-terminal region of Tome-1 was essential for binding to Skp-1, it was postulated that removing it might create a dominant negative form of the protein. To test this, recombinant ΔN-Tome-1 was purified and injected into both halves of dividing Xenopus embryos at the two-cell stage. After several cell divisions, it was observed that the embryos injected with ΔN-Tome-1 had a reduced number of cells relative to uninjected embryos. These data indicated that injection of ΔN-Tome-1 was affecting cell cycle progression, perhaps by inhibiting mitotic entry (FIG. 6A).

To determine whether ΔN-Tome-1 affected mitotic entry, the effects of adding ΔN-Tome-1 in vitro in Xenopus extracts were examined. Unfertilized egg extracts are arrested in meiosis by cytostatic factor (CSF) and can be induced to cycle into interphase and then into mitosis upon calcium addition (Murray (1991) Methods Cell Biol. 36:581). Cell cycle progression can be easily monitored in these extracts by observing nuclear formation and nuclear envelope breakdown of added sperm nuclei, the second being a direct measure of mitotic entry. To assay whether ΔN-Tome-1 affected mitotic entry in Xenopus egg extracts, wild-type and ΔN versions of Tome-1 were added to Xenopus mitotic (CSF) egg extracts released with calcium. The chromatin was visually examined to determine if nuclear formation or nuclear envelope breakdown had occurred in each case. As shown in FIG. 6B, incubation of these cycling egg extracts with 100 nM wild-type Tome-1 had no effect on the rate of mitotic entry 120 minutes after release from CSF arrest, as condensed nuclei were readily apparent. By contrast, incubation of the same Xenopus extract with 100 μM ΔN-Tome-1 mutant markedly inhibited mitotic entry, as the chromatin remained decondensed 120 minutes after calcium addition (FIG. 6B).

Since mitotic entry was inhibited when ΔN-Tome-1 was added to an extract, it was next determined whether ΔN-Tome-1 affected cdk1 activation, a critical step in entering mitosis. Cyclin bound cdk1 can oscillate between an active and inactive pool, with an inhibitory phosphorylation on tyrosine 15. To determine the activity of cdk1, Western blots were performed using a phosphotyrosine-specific anti-cdk1 antibody on samples taken 0, 60 or 120 minutes after release from CSF arrest. As shown in FIG. 6C, addition of buffer had no effect on the extent to which cdk1 was tyrosine phosphorylated. It was determined that there was no tyrosine phosphorylation on cdk1 at the 0 minute time point as expected, since cdk1 was active and there was no detectable inhibitory phosphorylation on tyrosine 15. The amount of tyrosine phosphorylation on cdk1 at the 60 minute time point, however, increased substantially as the extract cycled into interphase. As the extract entered mitosis, the amount of tyrosine phosphorylation on cdk1 decreased to the point of undetectability (FIG. 6C).

In contrast to what was observed above, addition of ΔN-Tome-1 greatly affected the amount of tyrosine phosphorylation on cdk1 relative to the buffer control. As expected, the amount of tyrosine phosphorylation on cdk1 at the 0 minute time point was low and high at the 60 minute time point. However, tyrosine phosphorylation on cdk1 remained high 120 minutes after release from CSF, indicating that ΔN-Tome-1 addition rendered cdk1 inactive and, consequently, inhibited mitotic entry.

Weel is one of the kinases known to mediate tyrosine phosphorylation of cdk1. Therefore, it was asked whether ΔN-Tome-1 inhibition of mitotic entry was due to wee 1. To test this, wee1 lwas immunodepleted from egg extracts mitotic entry was assayed in the presence of ΔN-Tome-1. It was determined that removing wee1 overrode the inhibition of mitotic entry observed in the presence of ΔN-Tome-1. As shown in FIG. 6D, while mitotic entry occurred in mock-depleted extracts, wee1 -depleted extracts and wee1 -depleted extracts supplemented with ΔN-tome-1, mitotic entry was blocked in mitotic extracts supplemented with ΔN-Tome-1.

### EXAMPLE V

### Tome-1 is Required for Mitotic Entry and Wee1 Degradation

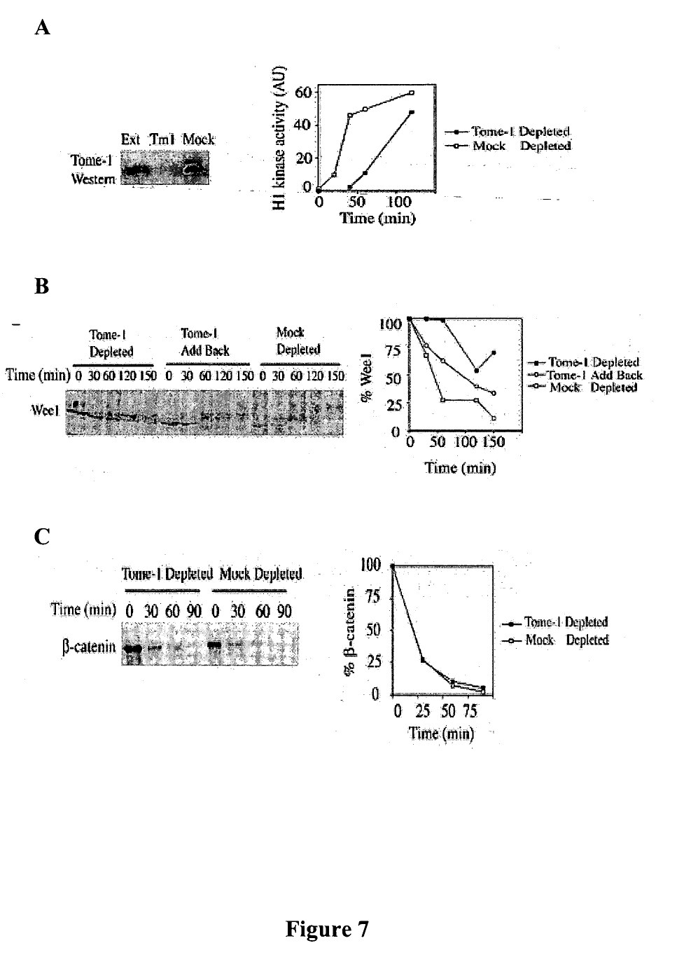

Although ΔN-Tome-1 inhibited mitotic entry in both embryos and extracts, it remained formally possible that the truncated form of the protein had some unexpected nonspecific effect. To address this issue, the effect of immunodepleting endogenous Tome-1 from extracts was measures. The consequence of depleting Tome-1 on cdk1 activity was measured by a conventional H1 kinase assay after releasing the extract from CSF arrest with Ca²⁺ for 20 minutes. FIG. 7A illustrates that greater than 95% of the endogenous Tome-1 was depleted with Dynal-beads containing the anti-Tome-1 antibody, while IgG control beads removed little or no Tome-1 from the extracts. Tome-1-depleted extracts supplemented with sperm nuclei entered mitosis much later than mock-depleted (IgG-depleted) extracts, indicating that Tome-1 is required for mitotic entry in Xenopus egg extracts (FIG. 7A).

Since higher levels of phosphotyrosine were observed on cdk1 (Y15) in the presence of ΔN-Tome-1 and since wee-1 is one of the kinases that phosphorylates cdk1 at this site, possible effects of removal of Tome-1 wee1 degradation were assayed. CSF extracts were incubated with either anti-Tome-1 antibody or control IgG antibody coupled to protein A-Dynal beads. After incubation for 2 hours at 4° C., sperm nuclei were added and the extracts were assayed for their capacity to degrade ³⁵S-labeled, in vitro translated wee1 after first releasing the extract from CSF arrest with Ca²⁺ for 20 minutes. As shown in FIG. 7B, the initial rate of wee1 degradation was inhibited 4- to 5-fold when Tome-1 was immunodepleted from Xenopus egg extracts. About half the degradation rate was recovered when Tome-1 was added back to the extract, indicating that Tome-1 is required for wee1 degradation. As a control, degradation of another SCF substrate, β-catenin, was assayed in the same extract. As depicted in FIG. 7C, degradation of β-catenin was unaffected when Tome-1 was immunodepleted from Xenopus egg extracts, indicating that the inability of wee1 to be degraded in Tome-1 -depleted extracts was not due to a general inhibition of degradation but, rather, a more specific effect on wee1.

Studies have indicated that that a feedback loop exists in which active cdk1 phosphorylates wee1, thereby leading to wee1 inactivation (Mueller et al. (1995) Mol. Biol. Cell 6:119). This mitotic phosphorylation of wee1 is easily observed by SDS-PAGE, since there is a substantial reduction of wee1 mobility (“mitotic shift”) at this time. Therefore, wee1 mobility can be used as a measure of whether an extract is in mitosis or interphase. As shown in FIG. 7B, wee1 does not shift appreciably in Tome-1-depleted extracts (some mitotic shifting is observed at the 120 minute and 150 minute time points upon overexposure of the film). By contrast, addition of Tome-1 back to the Tome1-depleted extract restored wee1 shifting at 60 minutes, indicating that this extract entered mitosis. Similarly, the mock-depleted extract contained mitotically shifted wee1 1 hour after the experiment was initiated, indicating that this extract cycled from interphase into mitosis.

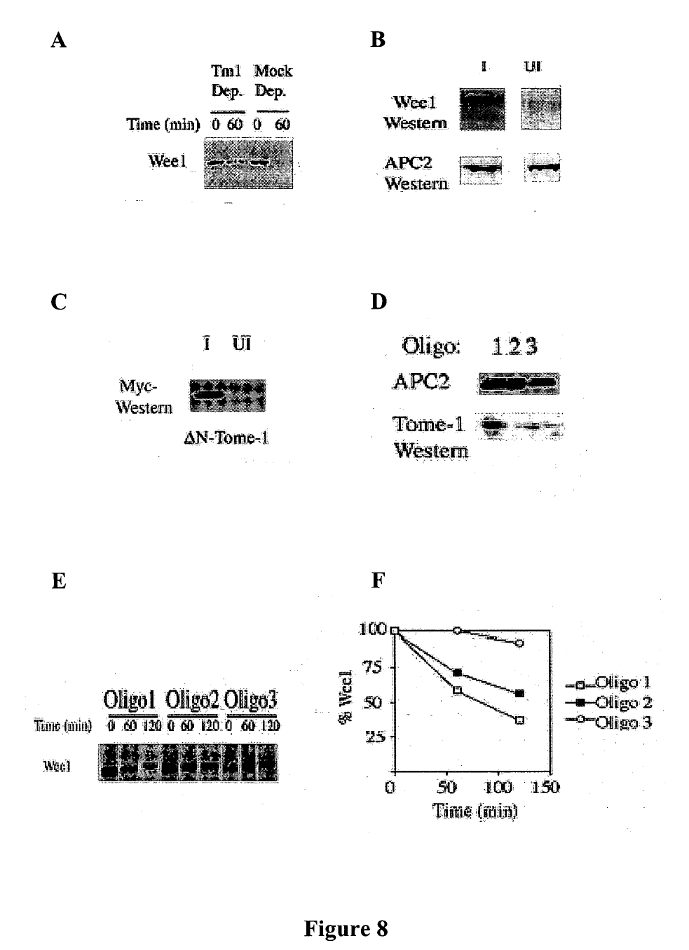

Recent studies have also indicated that wee1 degradation is accelerated in the presence of nuclei and that the nuclear pool of wee1 is particularly sensitive to proteolysis (Michael et al. (1998) Science 282:1886). To test if Tome1 is required for the nuclear-dependent degradation of wee1, the effect of removing Tome-1 on the degradation rate of nuclear wee1 was assayed. An experiment similar to the one described in FIG. 7B was performed, but the levels of wee1 in the nuclear fraction was measured after sedimenting nuclei through a sucrose cushion. It was insured that the nuclei remained intact throughout the experiment by preincubating the extract with cycloheximide, which inhibits the synthesis of cyclin B and mitotic entry, and subsequently checked nuclear morphology microscopically after Ca²⁺ addition (FIGS. 8A-8F). As shown in FIG. 8A, the nuclear pool of wee1 was completely degraded within 1 hour when Tome-1 was present, but was not substantially degraded when Tome-1 was depleted from the extract.

The above observations indicated that Tome-1 is required for the nuclear-dependent degradation of wee1 during an embryonic cell cycle. Studies have also demonstrated that wee1 is degraded in the somatic cell cycle (Watanabe et al. (1995) EMBO J. 14:1878). To examine the dependence of wee1 degradation on Tome-1 in somatic cells, an inducible cell line expressing a dominant negative version of mouse Tome-1 lacking the F box motif, ΔN-Tome-1 was engineered. As indicated in FIG. 8B, inducing expression of ΔN-Tome-1 increased the amount of wee1 present in 293 cells arrested with nocodazole by at least 10-fold. Without intending to be bound by theory, it was concluded from this experiment that expression of this mutant protein affected the wee1 degradation rate, rather than wee1 expression, since wee1 levels were assayed in nocodazole-arrested cells at a time when wee1 expression is not transcriptionally regulated (Kawasaki et al. (2001) EMBO J. 20:4618). Therefore, the expression of a dominant-negative form of Tome-1 in somatic cells inhibits wee1 degradation.

It was also determined that removing Tome-1 from somatic cells inhibited wee1 degradation. Tome-1 expression was reduced by transfecting 293 cells with small interfering RNA oligomers. Subsequently, wee1 degradation was assayed by pulse-chase analysis of endogenous wee1. As shown in FIG. 8D, wee1 degradation occurred in cells transfected with an oligomer that does not affect Tome-1 expression (Oligo1), but was inhibited in cells transfected with oligomers that affect Tome-1 expression (Oligo2 and Oligo3).

### EXAMPLE VI

### Tome-1 Associates with Phospho-Wee1 and is Required for its Ubiquitination

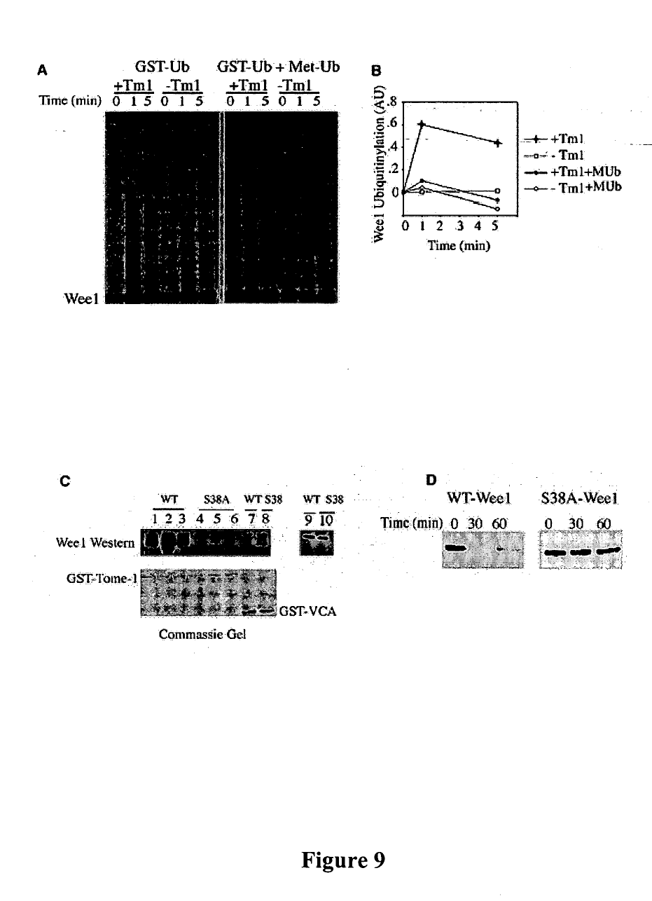

Since Tome-1 is required for wee1 degradation in both embryonic and somatic cells, possible requirements for wee1 ubiquitinylation were addressed. To test this, interphase Xenopus egg extracts were isolated and incubated them with sperm nuclei. Subsequently, in vitro translated wee1, GST-ubiquitin, or a mixture of GST-ubiquitin and methyl-ubiquitin (an effective inhibitor of polyubiquitinylation), were added (FIGS. 9A-9D). The extent of wee1 polyubiquitinylation in the presence or absence of excess Tome-1 was compared by auto-radiography of SDS-PAGE gels. As can be seen in FIG. 9A, including 50 nM Tome-1 in the reaction greatly increased the extent of wee1 ubiquitinylation. This observed ubiquitinylation was reduced in the presence of methyl-ubiquitin, thereby demonstrating that ubiquitinylation of wee1 was being measured as opposed to some other modification.

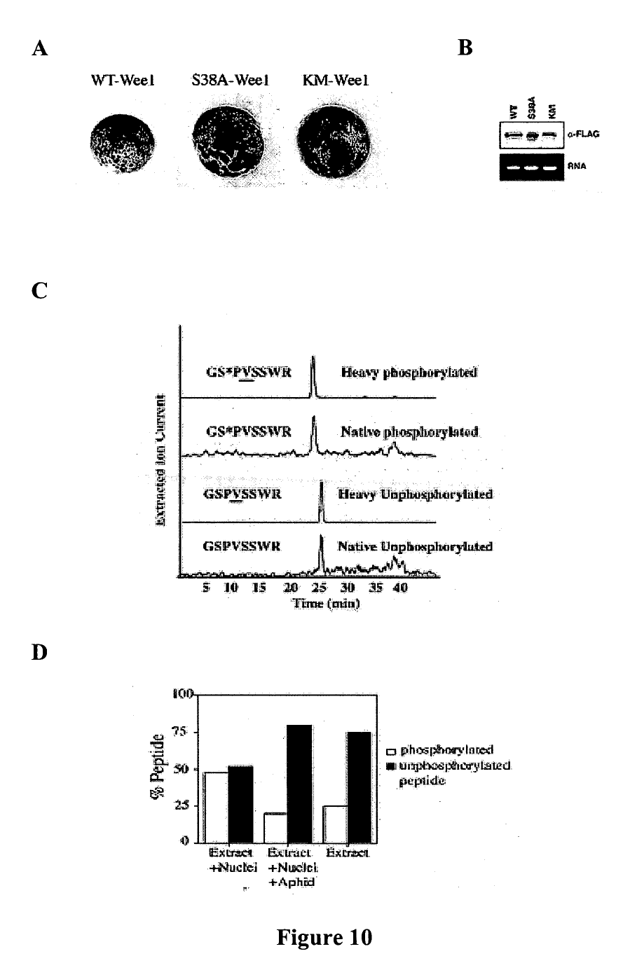

These ubiquitinylation studies indicated that Tome-1 was required for wee1 ubiquitinylation. Without intending to be bound by theory, one conclusion from these studies was that Tome-1 could interact with wee1 in a phospho-specific manner. To determine this, it was tested whether Tome-1 could interact with wee1 or a mutant of wee1 in which a serine was changed to alanine mutation at position 38, a site found to be phosphorylated in Xenopus interphase extracts (FIG. 10C). Wee1 or S38A wee1 was incubated with interphase egg extracts supplemented with nuclei and the proteasome inhibitor MG132. Subsequently, these extracts were incubated with GST-Tome-1 or an unrelated protein, GST-VCA. The amount of wee1 or S38A wee1 bound to the GST fusion proteins was determined by performing a glutathione precipitation assay and performing a Western blot for wee1 (FIG. 9C, lanes 1-3 for wild-type wee1 and lanes 4-6 for S38A wee1 ). As shown in FIG. 9C, GST-Tome-1 precipitated wild-type wee1 (25% of input wee1 was pulled down with GST-Tome-1 as opposed to 8% precipitated with GST-VCA). By contrast, the S38A mutant showed little interaction with GSTTome-1 (2.5% of input wee1 precipitated with GST-Tome-1 as compared to 8% precipitated with GST-VCA).

Without intending to be bound by theory, it was postulated that if phosphorylation of serine residue 38 in the N-terminal sequence of wee1 is necessary for interacting with Tome-1 and Tome-1 is required for degradation of wee1, mutating this site would dramatically affect the degradation rate of wee1. To test this hypothesis, the ability of wild-type wee1 and S38A-wee1 to be degraded in Xenopus interphase egg extracts supplemented with sperm nuclei was compared. As shown in FIG. 9D, the in vitro degradation rate of the S38A-wee1 mutant was much lower than the rate observed for wild-type wee1, indicating that phosphorylation at this site is critical for wee1 degradation. Consistent with this finding was the observation that injecting S38A-wee1 into dividing embryos inhibited mitotic entry (FIG. 10A). Without intending to be bound by theory, this inhibition of mitotic entry is likely due to the fact that in vivo S38A-wee1 was turned over more slowly than wild-type wee1 or a kinase inactive mutant of wee1 (FIG. 10B).

Without intending to be bound by theory, these studies indicated that the state of phosphorylation may be regulated physiologically and serves as an entry gate to mitotic progression. To test this idea, the phosphorylation levels on serine 38 were measured by a new mass spectrometry method that yields absolute quantitation of protein abundance and phosphorylation (Stemmann et al. (2001) Cell 107:715). This analysis indicated that the levels of wee1 phosphorylation depend on nuclei concentration. It was also determined that the extent of wee1 phosphorylation at serine 38 decreased by 60% when the DNA replication checkpoint was induced in the presence of nuclei and aphidicolin (FIGS. 10C and 10D). This was determined after mass spectrometric based quantification of wee1 isolated from interphase egg extracts, egg extracts supplemented with nuclei, or egg extracts supplemented with nuclei and aphidicolin. This analysis indicated that 48% of endogenous wee1 was phosphorylated in egg extracts supplemented with nuclei, 24% of wee1 was phosphorylated in egg extracts alone, and 20% of wee1 was phosphorylated in the presence of nuclei and aphidicolin (FIGS. 10C and 10D). There was no observable phosphorylation of this site in mitotic extracts.

### EXAMPLE VII

### Discussion

Tome-1 is a Substrate of the APC during G1

One of the striking characteristics of the cell cycle is that protein degradation acts as a trigger at several points to allow cell cycle transitions to occur. For example, the APC-mediated degradation of securin is required for cells to proceed from metaphase to anaphase (Cohen-Fix et al. (1996) Genes Dev. 10:3081), while the SCF-dependent degradation of cyclin-dependent kinase inhibitors is required to promote S phase (Peters (1998) Curr. Opin. Cell Biol. 10:759). The APC is also required to ubiquitinylate cyclin B in order to elicit mitotic exit. The APC then remains active until the end of G1, where SCF ligase activity begins to increase in order to help mediate entry into S phase. Proteolysis may also act to interweave different events in cell cycle progression. For example, the degradation of geminin by APC in mitosis assures that DNA replication can occur during the next S phase (McGarry et al. (1998) Cell 93:1043).

The SCF activators, the F box proteins, are also thought to be unstable proteins. One mode of regulation for these proteins has been proposed to be auto-ubiquitinylation (Zhou et al. (1998) Mol. Cell 2:571). In addition to this mode of regulation, without intending to be bound by theory a second, APC-dependent means of targeting F boxes for degradation is proposed. Based in part on the data presented herein, a general mechanism of tagging F boxes for degradation can exist since at least two other F box proteins are APC^{CDH1} substrates in vitro.

Tome-1 is Required for Mitotic Entry

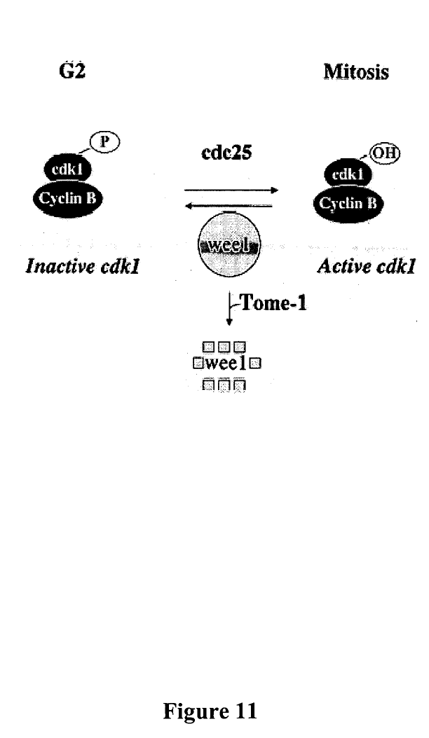

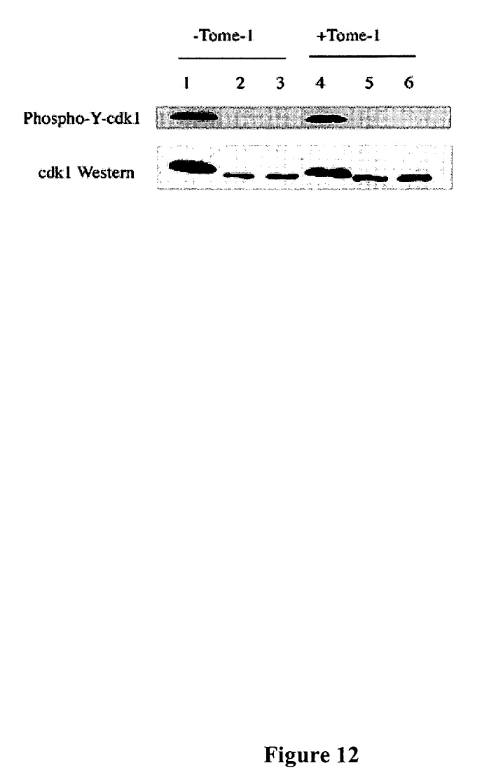

Without intending to be bound by theory, one model based in part on the data presented herein (FIG. 11) posits that Tome-1 mediates mitotic entry by targeting wee1 for degradation. Data presented herein demonstrate that Tome-1 has an F box and associates with Cul-1 and Skp-1. Data presented herein further demonstrate that Tome-1 associates with wee1 in a phosphospecific manner and that phosphorylation at this site is regulated during the cell cycle. An alternative view that Tome-1 is a phosphospecific inhibitor of wee1 has been eliminated by showing that Tome-1 has no effect on wee1 activity (see FIG. 12).

### EXAMPLE VIII

### Experimental Procedures

CDH1 Substrate Screen

Recombinant human CDH1 was purified from SF9 cells (Fang et al. (1998) Mol. Cell 2:163) and added to interphase egg extracts (Murray (1991) Methods Cell Biol. 36:581) to a final concentration of 0.4 nM (1 μl of 0.5 mg/ml CDH1 to 20 μl interphase extract). Pools of cDNAs from a Xenopus neurula stage library were in vitro translated in a Promega (Madison, Wis.) coupled translation/transcription system and individually added to CDH1 supplemented extracts at a ratio of 1:5 (in vitro translated product to egg extract). The same translation products were added to egg extracts lacking CDH1. Sample buffer was added to the degradation reactions after 1 hour. Positive pools containing proteins degraded specifically in the presence of CDH1 were isolated and the cDNA(s) corresponding to these proteins identified through a selection procedure described in Lustig et al. ((1997) Methods Enzymol. 283:83).

Antibody Production

The c DNA encoding Xenopus Tome-1 was subcloned into the pGEX4T-1 expression vector and the resulting construct transformed into BL-21 cells. The cells were induced with IPTG (0.5 mM) and the expressed fusion protein purified on glutathione beads. The protein was judged to be 90% pure by Coomassie blue staining and was sent to Zymed laboratories for immunization of rabbits. The resulting serum was then affinity purified using the Pierce Ab Purification System.

A peptide antibody corresponding to the C terminus of human Tome-1 (Zymed laboratories) was generated and used in Western analysis in FIGS. 3B and 8D.

Degradation Assays

Tome-1 degradation in Xenopus egg extracts was performed as previously described (Pfleger et al. (2000) Genes Dev. 14:655). 100 μM of MG132 or 100 μM of the N terminus of cyclin B were added to interphase egg extracts supplemented with 0.4 nM CDH1 to test their respective effects on Tome-1 degradation. Weel degradation assays were performed as described (Michael et al. (1998) Science 282:1886) with the following modifications. ³⁵S-labeled-IVT-wee1 was added to a CSF extract (2 μl wee1 to 25 μl Xenopus CSF egg extract) released from CSF with 0.8 mM CaCl₂. Degradation assays were initiated only after visual inspection of the added sperm nuclei indicated that interphase nuclei had formed. β-catenin degradation assays were performed as previously described (Salic et al. (2000) Mol. Cell 5:523). Ubiquitinylation assays were performed identically to degradation assays with the following exceptions: 30 mg/ml of GST-ubiquitin was added to interphase extracts in a 1:1 ratio while methyl-ubiquitin (Boston Biochem) was added to a final concentration of 10 mg/ml. The extent of wee1 poly-ubiquitinylation was measured by quantitating entire lanes after subtracting the signal generated from input wee1.

Tissue Culture and Cell Synchronization

The following protocol was used for cell synchrony experiments. HeLa cells were cultured in suspension in DMEM supplemented with 10% fetal calf serum and 100 μg/ml of penicillin/streptomycin at 37° C. Subsequently, thymidine (2 mM) was added to cells for 18 hours. The cells were then released from the thymidine arrest for 6 hours, at which time thymidine was added for an additional 12 hours. The cells were then harvested, and the thymidine was washed away to generate thymidine released cells. The thymidine released cells were then cultured for various times (0, 3.5, 6, 7, 8, 9 or 10 hours), harvested by centrifugation (500×g, 5 minutes), and lysed in SDS sample buffer.

An In Vitrogen ecdysone system was utilized to form an inducible form of ΔN-Tome-1. All transfections were performed using a standard calcium-phosphate method with 2 μg of DNA/4×10⁴ cells/ml. To test if myc-tagged Tome-1 and HA-tagged Skp-1 or Flag-Cul-1 interacted in tissue culture cells, cell lysates were prepared from transfected 293 cells in 25 mM Hepes (pH 7.7); 5 mM MgCl₂; 1 mM EDTA; 5 mM NaF; 0.1% Triton X-100; 250 mM sucrose; and 10 μg/ml of chymostatin, leupeptin, and pepstatin (Sigma). Cells were isolated after centrifugation, and a 1:1 ratio of lysis buffer:cell pellet was used. Cell lysates were made after dounce homogenization and clarified by centrifugation (14,000×g, 15 minutes). The clarified lysate was then added to 5 μl of anti-myc beads (Santa Cruz) for 1 hour at 4° C. The beads were subsequently isolated and successively washed in lysis buffer containing 100, 200 and 300 mM NaCl.

Immunofluorescence

NIH-3T3 cells were grown on coverslips in the presence of DMEM supplemented with 10% fetal calf serum and 100 μg/ml of penicillin/streptomycin at 37° C. The cells were fixed after incubation with 4% PFA in PBS for 10 minutes. They were subsequently incubated in PBS, 0.1% Triton X-100, 1% BSA, followed by primary and secondary antibodies in the same buffer. Primary (anti-Tome-1) and secondary antibodies (Texas-Red-anti-mouse from Jackson Labs) were used at dilutions of 1:100 and 1:500, respectively.

Immunodepletion

A previously described protocol was utilized in order to immunodeplete Tome-1 from CSF-arrested extracts (Funabiki et al. (2000) Cell 102:411). Briefly, 7 μg of affinity-purified anti-xTome-1 antibody was bound to 25 μl of protein-A conjugated Dyna beads (Dynal) for 1 hour at room temperature in PBS, 0.1% Triton X-100. The beads were then washed three times with PBS, 0.1% Triton X-100 and followed by three times with PBS, 0.5 M NaCl, 0.1%Triton X-100 and two times CSF-XB with 10 μg/ml each of chymostatin, leupeptin, and pepstatin and cytochalasin. The beads were then isolated with a Dynal magnet and added to 100 μl of CSF extract for 1 hour on ice. The depleted extract was then depleted again for an additional 1 hour on ice with another set of beads. The depleted extract was then used for wee1 degradation or mitotic entry (H1 kinase) assays. Tome-1 was added to a final concentration of 1 nM when added back to a depleted extract.

H1 Kinase Assays

H1 kinases were performed as described (Murray (1991) Methods Cell Biol. 36:581). Briefly, 16.5 μl of H1 kinase cocktail (EB buffer, 4 mM ATP, 5 mg/ml histone H1, and LPC) and 33 μl of EB buffer were added to 1.65 μl of Xenopus egg extract extracts on ice at the indicated times. The samples were then mixed, and the reactions were allowed to proceed at room temperature for 15 minutes, and subsequently stopped by adding 15 μl of the mix to 15 μl of 2× SDS-PAGE sample buffer. 20 μl of the stopped reaction were loaded on a 5%-15% PAGE gel and exposed to phosphorimager.

In Vitro Binding Assays

To assay association of Tome-1 with Skp-1, 5 μg of Tome-1 or ΔN-Tome- 1 was bound to 5 μl of glutathione beads and incubated in 100 μl of 1 mg/ml ovalbumin in XB buffer. 10 μl of in vitro translated, ³⁵S-labeled-Skp-1 was added to glutathione beads containing Tome-1 or GST alone for 1 hour at room temperature. The beads were isolated by centrifugation and washed three times in XB (100 μl), followed by one time in XB+0.3M NaCl. After a final wash in XB, the beads were isolated and SDS sample buffer was added to them. SDS-PAGE followed by phosphorimager analysis indicated the extent of binding of Tome-1 to Skp-1.

In vitro-Tome-1-wee1 binding assays: 50 nM GST-Tome-1 was added to Xenopus egg extracts (200 μl) in the presence of 200 sperm nuclei/μl and 10 μl of either in vitro translated wt-wee1 or S38A wee1. The amount of wee1 associated with Tome-1 or GST-VCA was determined after washing the beads three times with CSF-XB, SDS-PAGE and wee1 Western blot.

Identification of Serine 38 Phosphorylation

To identify phosphorylation of endogenous wee1, a protocol described in Murakami et al. ((1999) Genes Dev. 13:620) was utilized. The identity of the phosphorylation site was confirmed by mass spectrometry.

Mass Spectrometry

Silver-stained wee1 bands were excised from SDS-PAGE gels. After addition of the ¹³C-labeled non-phosphorylated and phosphorylated peptides as internal standards, the samples were in-gel reduced, alkylated, and digested as described previously (Shevchenko et al. (1996) Anal. Chem. 68:850). In order to account for the reduced digestion efficiency of trypsin when phosphoserine residues are in the +2 position relative to a lysine or arginine residue (Schlosser et al. (2001) Anal. Chem. 73:170). NEGPQKG(pS)PVSSWRTNN (SEQ ID NO:7) (a gift from Cell Signaling Technology) was used as internal standard (the underlined valine residue was ¹³CX6-labeled). Extracted peptides were analyzed by LC/MS using an LCQ-DECA ion trap mass spectrometer (Thermo Finnigan, San Jose, Calif.). Quantitation of wee1 was performed as described previously (Stemmann et al. (2001) Cell 107:715).

### EXAMPLE IX

### Chromosome 12p13

A recent seminar presented data that demonstrated a recurring problem with transplantation of pluripotent stem cells is the fact that tumors arise very frequently post-transplantation (Gearhart (December 2003) “Differentiation and Transplantation of Human Pluripotent Stem Cells.” American Society Meeting). Data were presented that in these mouse tumors, there is frequent over-amplification of genes in chromosome 12p13. Interestingly, this region contains several genes, one of which encodes Tome-1. Therefore, Tome-1 amplification could be a hallmark of tumorigenesis. Accordingly, embodiments of the present invention are directed to screening tumor samples for tome-1 overexpression as a prognostic indicator.

### EQUIVALENTS

Other embodiments will be evident to those of skill in the art. It should be understood that the foregoing description is provided for clarity only and is merely exemplary. The spirit and scope of the present invention are not limited to the above examples, but are encompassed by the following claims. All publications and patent applications cited above are incorporated by reference herein in their entirety for all purposes to the same extent as if each individual publication or patent application were specifically indicated to be so incorporated by reference.

1. An isolated nucleic acid molecule which encodes a polypeptide comprising an amino acid sequence having at least about 60% sequence homology to an amino acid sequence selected from the group consisting of SEQ ID NO:1, SEQ ID NO:2 and SEQ ID NO:3, wherein the polypeptide has one or more Tome-1 activities.

2. The polypeptide of claim 1, wherein the Tome-1 activities are selected from the group consisting of modulating wee1 ubiquitinylation, modulating wee1 degradation, modulating an SCF complex component activity and modulating mitotic entry.

3. An isolated nucleic acid molecule which encodes a polypeptide, wherein the nucleic acid molecule encodes a nucleotide sequence having at least about 60% sequence homology to a nucleic acid comprising a nucleotide sequence selected from the group consisting of SEQ ID NO:4, SEQ ID NO:5 and SEQ ID NO:6, and wherein the polypeptide has one or more Tome-1 activities.

4. An isolated nucleic acid molecule comprising a nucleotide sequence selected from the group consisting of SEQ ID NO:4, SEQ ID NO:5 and SEQ ID NO:6.

5. An isolated nucleic acid molecule comprising a nucleotide sequence having at least about 85% sequence homology to a nucleotide sequence selected from the group consisting of SEQ ID NO:4, SEQ ID NO:5 and SEQ ID NO:6, or a complement thereof.

6. An isolated nucleic acid molecule comprising a nucleic acid molecule which encodes a polypeptide comprising an amino acid sequence having at least about 85% sequence homology to an amino acid sequence selected from the group consisting of SEQ ID NO:1, SEQ ID NO:2 and SEQ ID NO:3.

7. An isolated nucleic acid molecule, which hybridizes to a complement of the nucleic acid molecule of any one of claims 1, 3, 4, 5 or 6.

8. An isolated nucleic acid molecule comprising a nucleotide sequence which is complementary to the nucleotide sequence of the nucleic acid molecule of any one of claims 1, 3, 4, 5 or 6.

9. An isolated nucleic acid molecule of any one of claims 1, 3, 4, 5 or 6, further comprising a nucleotide sequence encoding a heterologous polypeptide.

10. A vector comprising the nucleic acid molecule of any one of claims 1, 3, 4, 5 or 6.

11. The vector of claim 10, which is an expression vector.

12. A host cell transfected with the expression vector of claim 11.

13. A method of producing a polypeptide comprising culturing the host cell of claim 12 in an appropriate culture medium to, thereby, produce the polypeptide.

14. An isolated polypeptide comprising an amino acid sequence having at least about 60% sequence homology to an amino acid sequence selected from the group consisting of SEQ ID NO:1, SEQ ID NO:2 and SEQ ID NO:3, wherein the polypeptide has one or more Tome-1 activities.

15. The isolated polypeptide of claim 14, wherein the Tome-1 activities are selected from the group consisting of modulating wee1 ubiquitinylation, modulating wee1 degradation, modulating an SCF complex component activity and modulating mitotic entry.

16. An isolated polypeptide comprising the amino acid sequence selected from the group consisting of SEQ ID NO:1, SEQ ID NO:2 and SEQ ID NO:3.

17. An isolated polypeptide which is encoded by a nucleic acid molecule comprising a nucleotide sequence having at least about 85% sequence homology to a nucleotide sequence selected from the group consisting of SEQ ID NO:4, SEQ ID NO:5 and SEQ ID NO:6, or a complement thereof.

18. An isolated polypeptide comprising an amino acid sequence having at least about 85% sequence homology to an amino acid sequence selected from the group consisting of SEQ ID NO:1, SEQ ID NO:2 and SEQ ID NO:3.

19. The polypeptide of any one of claims 14, 16, 17 or 18, further comprising heterologous amino acid sequences.

20. An antibody which selectively binds to a polypeptide of any one of claims 14, 16, 17 or 18.

21. A method for detecting a polypeptide of any one of claims 14, 16, 17 or 18 in a sample comprising the steps of:
a) contacting the sample with a compound which selectively binds to the polypeptide; and
b) determining whether the compound binds to the polypeptide in the sample to thereby detect the presence of the polypeptide in the sample.

22. The method of claim 21, wherein the compound which binds to the polypeptide is an antibody.

23. A kit comprising a compound which selectively binds to a polypeptide of any one of claims 14, 16, 17 or 18 and instructions for use.

24. A method for detecting the nucleic acid molecule of any one of claims 1, 3, 4, 5 or 6 in a sample comprising the steps of:
a) contacting the sample with a nucleic acid probe or primer which selectively hybridizes to a complement of the nucleic acid molecule; and
b) determining whether the nucleic acid probe or primer binds to the complement of the nucleic acid molecule in the sample to thereby detect the presence of the nucleic acid molecule in the sample.

25. The method of claim 24, wherein the sample comprises mRNA molecules and is contacted with a nucleic acid probe.

26. A kit comprising a compound which selectively hybridizes to a complement of the nucleic acid molecule of any one of claims 1, 3, 4, 5 or 6 and instructions for use.

27. A method for identifying a compound which binds to a polypeptide of any one of claims 14, 16, 17 or 18 comprising the steps of:
a) contacting the polypeptide with a test compound; and
b) determining whether the polypeptide binds to the test compound.

28. The method of claim 27, wherein the binding of the test compound to the polypeptide is detected by a method selected from the group consisting of detection of binding by direct detection of test compound/polypeptide binding; detection of binding using a competition binding assay; and detection of binding using an assay for a Tome-1 activity.

29. The method of claim 28, wherein the Tome-1 activity is selected from the group consisting of modulating wee1 ubiquitinylation, modulating wee1 degradation, modulating an SCF complex component activity and modulating mitotic entry.

30. A method for modulating a Tome-1 activity of a polypeptide of any one of claims 14, 16, 17 or 18 comprising contacting the polypeptide with an effective amount of a compound to modulate the Tome-1 activity of the polypeptide.

31. A method for identifying a compound that modulates a Tome-1 activity of a polypeptide of any one of claims 14, 16, 17 or 18 comprising the steps of:
a) contacting the polypeptide with a test compound; and
b) determining a modulation of a Tome-1 activity of the polypeptide, thereby identifying a compound that modulates the activity.

32. The method of claim 31, wherein the Tome-1 activity is selected from the group consisting of modulating wee1 ubiquitinylation, modulating wee1 degradation, modulating an SCF complex component activity and modulating mitotic entry.

33. A method for identifying a compound that modulates mitosis comprising the steps of:
a) contacting a polypeptide of any one of claims 14, 16, 17 or 18 with the compound; and
b) determining a modulation of a Tome-1 activity, thereby identifying a compound that modulates mitosis.

34. The method of claim 33, wherein the Tome-1 activity is selected from the group consisting of modulating wee1 ubiquitinylation, modulating wee1 degradation, modulating an SCF complex component activity and modulating mitotic entry.

35. A method of modulating mitosis in a subject comprising administering to the subject a therapeutically effective amount of a compound identified in claim 33.

36. The method of claim 35, wherein the subject is a human.

37. The method of claim 35, wherein the compound is an antibody.

38. The method of claim 35, wherein the compound is an antisense molecule.

39. The method of claim 35, wherein the compound is a peptide.

40. The method of claim 35, wherein the compound is a small molecule.

41. The method of claim 35, wherein the compound inhibits mitosis.

42. The method of claim 35, wherein the compound enhances mitosis.

43. A method of modulating mitosis comprising contacting a polypeptide of any one of claims 16, 17 or 18 with an effective amount of a compound to modulate mitosis.

44. A method of modulating mitosis in a cell comprising contacting a cell expressing a polypeptide of any one of claims 14, 16, 17 or 18 with an effective amount of a compound to modulate mitosis in the cell.

45. A method of modulating mitosis in a subject comprising administering to the subject a therapeutically effective amount of a nucleic acid of any one of claims 1, 3, 4, 5 or 6.

46. The method of claim 45, wherein the subject is a human.

47. A method for identifying a compound that modulates a Tome-1 activity of a polypeptide of any one of claims 14, 16, 17 or 18, wherein the polypeptide is expressed in a cell, comprising the steps of:
a) contacting a cell expressing the polypeptide with a test compound; and
b) determining a modulation of a Tome-1 activity, thereby identifying a compound that modulates the activity.

48. The method of claim 47, wherein the activity is selected from the group consisting of modulating wee1 ubiquitinylation, modulating wee1 degradation, modulating SCF complex component activity and modulating mitotic entry.

49. A method of treating a cellular proliferative disorder in subject in need thereof, the method comprising administering to the subject a compound that modulates a Tome-1 activity.

50. The method of claim 49, wherein Tome-1 expression is inhibited.

51. The method of claim 49, wherein said cellular proliferative disorder is cancer.
